SEQUENCE LISTING (52 sequences)

SEQ ID NO: 1 - DNA (genomic), 1431 bp; single-stranded, linear
  units: bp
  authors: Drayna, Dennis Jarnagin, Alisha Stephens McLean, John Henzel, William Kohr, William Fielding, Christopher Lawn, Richard
  title: Cloning and sequencing of human cholesteryl ester transfer protein cDNA
  journal: Nature
  volume: 327
  pages: 632-634
  date: June 18-1987
tgctccaaaggcacctcgcacgaggcaggcatcgtgtgccgcatcaccaagcctgccctcctggtgttgaaccacgagactgccaaggtcatccagaccgccttccagcgagccagctacccagatatcacgggcgagaaggccatgatgctccttggccaagtcaagtatgggttgcacaacatccagatcagccacttgtccatcgccagcagccaggtggagctggtggaagccaagtccattgatgtctccattcagaacgtgtctgtggtcttcaaggggaccctgaagtatggctacaccactgcctggtggctgggtattgatcagtccattgacttcgagatcgactctgccattgacctccagatcaacacacagctgacctgtgactctggtagagtgcggaccgatgcccctgactgctacctgtctttccataagctgctcctgcatctccaaggggagcgagagcctgggtggatcaagcagctgttcacaaatttcatctccttcaccctgaagctggtcctgaagggacagatctgcaaagagatcaacgtcatctctaacatcatggccgattttgtccagacaagggctgccagcatcctttcagatggagacattggggtggacatttccctgacaggtgatcccgtcatcacagcctcctacctggagtcccatcacaagggtcatttcatctacaagaatgtctcagaggacctccccctccccaccttctcgcccacactgctgggggactcccgcatgctgtacttctggttctctgagcgagtcttccactcgctggccaaggtagctttccaggatggccgcctcatgctcagcctgatgggagacgagttcaaggcagtgctggagacctggggcttcaacaccaaccaggaaatcttccaagaggttgtcggcggcttccccagccaggcccaagtcaccgtccactgcctcaagatgcccaagatctcctgccaaaacaagggagtcgtggtcaattcttcagtgatggtgaaattcctctttccacgcccagaccagcaacattctgtagcttacacatttgaagaggatatcgtgactaccgtccaggcctcctattctaagaaaaagctcttcttaagcctcttggatttccagattacaccaaagactgtttccaacttgactgagagcagctccgagtccatccagagcttcctgcagtcaatgatcaccgctgtgggcatccctgaggtcatgtctcggctcgaggtagtgtttacagccctcatgaacagcaaaggcgtgagcctcttcgacatcatcaaccctgagattatcactcgagatggcttcctgctgctgcagatggactttggcttccctgagcacctgctggtggatttcctccagagcttgagctag

SEQ ID NO: 2 - protein, 20 aa
EIFQELSRGLPTGQAQVAVH

SEQ ID NO: 3 - protein, 20 aa
VAVTFRFPRPDGREAVAYRF

SEQ ID NO: 4 - protein, 22 aa
LLLQMDFGFPKHLLVDFLQSLS

SEQ ID NO: 5 - protein, 20 aa
TTVQASYSQKKLFLHLLDFQ

SEQ ID NO: 6 - protein, 20 aa
LLLHLQGEREPGWLKQLFTN

SEQ ID NO: 7 - protein, 20 aa
DVSGERAVMLLGRVKYGLHN

SEQ ID NO: 8 - protein, 20 aa
QEIFQEVVGGFPSQAQVTVH

SEQ ID NO: 9 - protein, 20 aa
VMVKFLFPRPDQQHSVAYTF

SEQ ID NO: 10 - protein, 22 aa
LLLQMDFGFPEHLLVDFLQSLS

SEQ ID NO: 11 - protein, 20 aa
TTVQASYSKKKLFLSLLDFQ

SEQ ID NO: 12 - protein, 20 aa
LLLHLQGEREPGWIKQLFTN

SEQ ID NO: 13 - protein, 20 aa
DITGEKAMMLLGQVKYGLHN

SEQ ID NO: 14 - DNA (genomic), 63 bp; single-stranded, linear
  units: bp
caggaaatcttccaggagctttccagaggccttcccaccggccaggcccaggtagccgtccac

SEQ ID NO: 15 - DNA (genomic), 60 bp; single-stranded, linear
  units: bp
gtcgccgtgacgttccgcttcccccgcccagatggccgagaagctgtggcctacaggttt

SEQ ID NO: 16 - DNA (genomic), 66 bp; single-stranded, linear
  units: bp
ctgctgctgcagatggacttcggttttcccaagcacctgctggtggatttcctgcagagcctgagc

SEQ ID NO: 17 - DNA (genomic), 60 bp; single-stranded, linear
  units: bp
accaccgtccaggcctcctactcccagaaaaagctcttcctacacctcttggatttccag

SEQ ID NO: 18 - DNA (genomic), 60 bp; single-stranded, linear
  units: bp
ctgctcctgcacctccagggggagcgcgagccggggtggctcaagcagctcttcacaaac

SEQ ID NO: 19 - DNA (genomic), 60 bp; single-stranded, linear
  units: bp
gacgtcagcggcgagagggccgtgatgctcctcggccgggtcaagtacgggctgcacaac

SEQ ID NO: 20 - DNA (genomic), 63 bp; single-stranded, linear
  units: bp
caggaaatcttccaagaggttgtcggcggcttccccagccaggcccaagtcaccgtccactgc

SEQ ID NO: 21 - DNA (genomic), 60 bp; single-stranded, linear
  units: bp
gtgatggtgaaattcctctttccacgcccagaccagcaacattctgtagcttacacattt

SEQ ID NO: 22 - DNA (genomic), 66 bp; single-stranded, linear
  units: bp
ctgctgctgcagatggactttggcttccctgagcacctgctggtggatttcctccagagcttgagc

SEQ ID NO: 23 - DNA (genomic), 60 bp; single-stranded, linear
  units: bp
actaccgtccaggcctcctattctaagaaaaagctcttcttaagcctcttggatttccag

SEQ ID NO: 24 - DNA (genomic), 60 bp; single-stranded, linear
  units: bp
ctgctcctgcatctccaaggggagcgagagcctgggtggatcaagcagctgttcacaaat

SEQ ID NO: 25 - DNA (genomic), 60 bp; single-stranded, linear
  units: bp
gatatcacgggcgagaaggccatgatgctccttggccaagtcaagtatgggttgcacaac

SEQ ID NO: 26 - protein, 497 aa
  authors: Nagashima, M. McLean, J. W. Lawn, R. M.
  title: Cloning and mRNA tissue distribution of rabbit cholesteryl ester transfer protein
  journal: J. Lipid Res.
  volume: 29
  pages: 1643-1649
  date: 1988
ACPKGASYEAGIVCRITKPALLVLNQETAKVVQTAFQRAGYPDVSGERAVMLLGRVKYGLHNLQISHLSIASSQVELVDAKTIDVAIQNVSVVFKGTLNYSYTSAWGLGINQSVDFEIDSAIDLQINTELTCDAGSVRTNAPDCYLAFHKLLLHLQGEREPGWLKQLFTNFISFTLKLILKRQVCNEINTISNIMADFVQTRAASILSDGDIGVDISVTGAPVITATYLESHHKGHFTHKNVSEAFPLRAFPPGLLGDSRMLYFWFSDQVLNSLARAAFQEGRLVLSLTGDEFKKVLETQGFDTNQEIFQELSRGLPTGQAQVAVHCLKVPKISCQNRGVVVSSSVAVTFRFPRPDGREAVAYRFEEDIITTVQASYSQKKLFLHLLDFQCVPASGRAGSSANLSVALRTEAKAVSNLTESRSESLQSSLRSLIATVGIPEVMSRLEVAFTALMNSKGLDLFEIINPEIITLDGCLLLQMDFGFPKHLLVDFLQSLS

SEQ ID NO: 27 - DNA (genomic), 1494 bp; single-stranded, linear
  units: bp
  authors: Nagashima, Mariko McLean, John W. Lawn, Richard M.
  title: Cloning and mRNA tissue distribution of rabbit cholesteryl ester transfer protein
  journal: J. Lipid Res.
  volume: 29
  pages: 1643-1649
  date: 1988
gcctgtcccaaaggcgcctcctacgaggctggcatcgtgtgtcgcatcaccaagcccgccctcttggtgttgaaccaagagacggccaaggtggtccagacggccttccagcgcgccggctatccggacgtcagcggcgagagggccgtgatgctcctcggccgggtcaagtacgggctgcacaacctccagatcagccacctgtccatcgccagcagccaggtggagctggtggacgccaagaccatcgacgtcgccatccagaacgtgtccgtggtcttcaaggggaccctgaactacagctacacgagtgcctgggggttgggcatcaatcagtctgtcgacttcgagatcgactctgccattgacctccagatcaacacagagctgacctgcgacgctggcagtgtgcgcaccaatgcccccgactgctacctggctttccataaactgctcctgcacctccagggggagcgcgagccggggtggctcaagcagctcttcacaaacttcatctccttcaccctgaagctgattctgaagcgacaggtctgcaatgagatcaacaccatctccaacatcatggctgactttgtccagacgagggccgccagcatcctctcagatggagacatcggggtggacatttccgtgacgggggcccctgtcatcacagccacctacctggagtcccatcacaagggtcacttcacgcacaagaacgtctccgaggccttccccctccgcgccttcccgcccggtcttctgggggactcccgcatgctctacttctggttctccgatcaagtgctcaactccctggccagggccgccttccaggagggccgtctcgtgctcagcctgacaggggatgagttcaagaaagtgctggagacccagggtttcgacaccaaccaggaaatcttccaggagctttccagaggccttcccaccggccaggcccaggtagccgtccactgccttaaggtgcccaagatctcctgccagaaccggggtgtcgtggtgtcttcttccgtcgccgtgacgttccgcttcccccgcccagatggccgagaagctgtggcctacaggtttgaggaggatatcatcaccaccgtccaggcctcctactcccagaaaaagctcttcctacacctcttggatttccagtgcgtgccggccagcggaagggcaggcagctcagcaaatctctccgtggccctcaggactgaggctaaggctgtttccaacctgactgagagccgctccgagtccctgcagagctctctccgctccctgatcgccacggtgggcatcccggaggtcatgtctcggctcgaggtggcgttcacagccctcatgaacagcaaaggcctggacctcttcgaaatcatcaaccccgagattatcactctcgatggctgcctgctgctgcagatggacttcggttttcccaagcacctgctggtggatttcctgcagagcctgagctag

SEQ ID NO: 28 - protein, 476 aa
  authors: Drayna, Dennis Jarnagin, Alisha Stephens McLean, John Henzel, William Kohr, William Fielding, Christopher Lawn, Richard
  title: Cloning and sequencing of human cholesteryl ester transfer protein cDNA
  journal: Nature
  volume: 327
  pages: 632-634
  date: June 18-1987
CSKGTSHEAGIVCRITKPALLVLNHETAKVIQTAFQRASYPDITGEKAMMLLGQVKYGLHNIQISHLSIASSQVELVEAKSIDVSIQNVSVVFKGTLKYGYTTAWWLGIDQSIDFEIDSAIDLQINTQLTCDSGRVRTDAPDCYLSFHKLLLHLQGEREPGWIKQLFTNFISFTLKLVLKGQICKEINVISNIMADFVQTRAASILSDGDIGVDISLTGDPVITASYLESHHKGHFIYKNVSEDLPLPTFSPTLLGDSRMLYFWFSERVFHSLAKVAFQDGRLMLSLMGDEFKAVLETWGFNTNQEIFQEVVGGFPSQAQVTVHCLKMPKISCQNKGVVVNSSVMVKFLFPRPDQQHSVAYYFEEDIVTTVQASYSKKKLFLSLLDFQITPKTVSNLTESSSESIQSFLQSMITAVGIPEVMSRLEVVFTALMNSKGVSLFDIINPEIITRDGFLLLQMDFGFPEHLLVDFLQSLS

SEQ ID NO: 29 - protein, 26 aa
  authors: Swenson, T. L. et al.,
  journal: J. Biol. Chem.
  volume: 264
  pages: 14318-14326
  date: 1989
RDGFLLLQMDFGFPEHLLVDFLQSLS

SEQ ID NO: 30 - protein, 493 aa
  authors: Pape, Michael E. Rehberg, Edward F. Marotti, Keith R. Melchior, George W.
  title: Molecular Cloning, Sequence, and Expression of Cynomolgus Monkey Cholesteryl Ester Transfer Protein
  journal: Arteriosclerosis and Thrombosis
  volume: 11
  issue: 6
  pages: 1759-1771
  date: Nov/Dec-1991
MLAATVLTLALLGNVHACSKGTSHKAGIVCRITKPALLVLNQETAKVIQSAFQRANYPNITGEKAMMLLGQVKYGLHNIQISHLSIASSRVELVEAKSIDVSIQNVSVVFKGTLKYGYTTAWGLGIDQSVDFEIDSAIDLQINTQLTCDSGRVRTDAPDCYLSFHKLLLHLQGEREPGWIKQLFTNFISFTLKLVLKGQICKEINIISNIMADFVQTRAASILSDGDIGVDISLTGDPIITASYLESHHKGYFIYKNVSEDLPLPTFSPALLGDSRMLYFWFSEQVFHSLAKVAFQDGRLTLSLMGDEFKAVLETWGFNTNQEIFQEVVGGFPSQAQVTVHCLKMPRISCQNKGVVVNSSVMVKFLFPRPDQQHSVAYTFEEDIMTTVQASYSKKKLFLSLLDFQITPKTVSNLTESSSESVQSFLQSMITTVGIPEVMSRLEAVFTALMNSKGLSLFDIINPEIITRDGFLLLQMDFGFPEHLLVDFLQSLS

SEQ ID NO: 31 - DNA (genomic), 1508 bp; single-stranded, linear
  authors: Pape, Michael E. Rehberg, Edward F. Marotti, Keith R. Melchior, George W.
  title: Molecular Cloning, Sequence, and Expression of Cynomolgus Monkey Cholesteryl Ester Transfer Protein
  journal: Arteriosclerosis and Thrombosis
  volume: 11
  issue: 6
  pages: 1759-1771
  date: Nov/Dec-1991
atgctggctgccaccgtcctgaccctggccctgctgggcaatgtccacgcctgctccaaaggtacctcacacaaggcaggcattgtgtgccgcatcaccaagcctgccctcctggtgttgaaccaacagactgccaaggtgatccagtctgccttccagcgagccaactacccaaatatcacaggcgagaaggccatgatgctccttggccaagtcaagtatgggttgcacaacatccaaatcagccacttgtccatcgccagcagccgggtggagctggtggaagccaagtccattgatgtctccattcagaacgtgtctgtggtcttcaaggggaccctgaagtatggctacaccactgcctgggggctgggcattgatcagtccgttgacttcgagatcgactctgccattgacctccagatcaacacacaactgacctgtgactctggtagagtgaggactgatgcccctgactgctacctgtctttccataagctgctcctgcatctccaaggggagcgagagcccgggtggatcaagcagctgttcacaaacttcatctccttcaccctgaagctggtcctgaagggacagatctgcaaagagatcaacatcatctccaacatcatggccgattttgtccagacaagggctgccagtatcctttcagatggagacatcggggtggacatttccctgacaggtgatcccatcattacagcctcctacctggagtcccatcacaagggttatttcatctataagaatgtctcggaggacctcccactccccaccttctcgcccgcactgctgggggactcccgcatgctgtacttctggttctccgagcaagtcttccactccctggccaaggtagctttccaagatgcccgcctcacgctcagcctgatgggagacgagttcaaggcagtgctggagacctggggcttcaacaccaaccaagaaatcttccaggaggttgtcggcggcttccccagccaggcccaagtcaccgtccactgcctcaagatgcccaggatctcctgccaaaacaagggagtcgtggtcaattcttcggtgatggtgaaattcctctttccacgcccagaccagcaacactctgtagcttacacatttgaagaggatatcatgaccaccgtccaggcctcctattctaagaaaaagctcttcttaagcctcttggatttccagattacaccaaagactgtttccaacttgactgagagcagctccgagtccgtccagagcttcctgcagtcaatgatcaccactgtgggcatccctgaggtcatgtctcggcttgaggcagtgtttacagccctcatgaacagcaaaggcctgagcctcttcgacatcatcaatcctgagattatcactcgagatggcttcctgctgctgcagatggactttggcttccctgagcacctgctggtggatttcctccagagcttgagctagaagtctccaaggacgtcaggatgggg

SEQ ID NO: 32 - protein, 20 aa
QEIFQEVVGGFPSQAQVTVH

SEQ ID NO: 33 - protein, 20 aa
VMVKFLFPRPDQQHSVAYTF

SEQ ID NO: 34 - protein, 22 aa
LLLQMDFGFPEHLLVDFLQSLS

SEQ ID NO: 35 - protein, 20 aa
TTVQASYSKKKLFLSLLDFQ

SEQ ID NO: 36 - protein, 20 aa
LLLHLQGEREPGWIKQLFTN

SEQ ID NO: 37 - protein, 20 aa
NITGEKAMMLLGQVKYGLHN

SEQ ID NO: 38 - protein, 183 aa
MDIDPYKEFGATVELLSFLPSDFFPSVRDLLDTASALYREALESPEHCSPHHTALRQAILCWGELMTLATWVGVNLEDPASRDLVVSYVNTNMGLKFRQLLWFHISCLTFGRETVIEYLVSFGVWIRTPPAYRPPNAPILSTLPETTVVRRRGRSPRRRTPSPRRRRSQSPRRRRSQSRESQC

SEQ ID NO: 39 - DNA (genomic), 552 bp; single-stranded, linear
atggacatcgacccttataaagaatttggagctactgtggagttactctcgtttttgccttctgacttctttccttcagtacgagatcttctagataccgcctcagctctgtatcgggaagccttagagtctcctgagcattgttcacctcaccatactgcactcaggcaagcaattctttgctggggggaactaatgactctagctacctgggtgggtgttaatttggaagatccagcgtctagagacctagtagtcagttatgtcaacactaatatgggcctaaagttcaggcaactcttgtggtttcacatttcttgtctcacttttggaagagaaacagttatagagtatttggtgtctttcggagtgtggattcgcactcctccagcttatagaccaccaaatgcccctatcctatcaacacttccggagactactgttgttagacgacgaggcaggtcccctagaagaagaactccctcgcctcgcagacgaaggtctcaatcgccgcgtcgcagaagatctcaatctcgggaatctcaatgttag

SEQ ID NO: 40 - protein, 25 aa
TWVGVNLEDPASRDLVVSYVNTNMG

SEQ ID NO: 41 - protein, 21 aa
LLWFHISCLTFGRETVIEYLV

SEQ ID NO: 42 - protein, 16 aa
VVSYVNTNMGLKFRQL

SEQ ID NO: 43 - protein, 21 aa
VSFGVWIRTPPAYRPPNAPIL

SEQ ID NO: 44 - DNA (cDNA), 35 bp; single-stranded, linear
gatcccatggacatcgacccttataaagaatttgg

SEQ ID NO: 45 - DNA (cDNA), 44 bp; single-stranded, linear
gatcaagcttttaacattgagattcccgagattgagatcttctg

SEQ ID NO: 46 - DNA (cDNA), 43 bp; single-stranded, linear
gatcgaattcactagttggaagatccagcgtctagagacctag

SEQ ID NO: 47 - DNA (cDNA), 41 bp; single-stranded, linear
gatcgaattcctcgagctagagtcattagttccccccagca

SEQ ID NO: 48 - DNA (cDNA), 34 bp; single-stranded, linear
gattatcactcgagatggcttcctgctgctgcag

SEQ ID NO: 49 - DNA (cDNA), 40 bp; single-stranded, linear
gatcgaattcagcgctcaagctctggaggaaatccaccag

SEQ ID NO: 50 - protein, 26 aa
LDGCLLLQMDFGFPKHLLVDFLQSLS

SEQ ID NO: 51 - DNA (cDNA), 51 bp; single-stranded, linear
ggccgcaacgttttactagctcaggctctgcaggaaatccaccagcaggtg

SEQ ID NO: 52 - DNA (cDNA), 41 bp; single-stranded, linear
ggccgcccatggcctgtcccaaaggcgcctcctacgaggct

(19) United States
(12) Patent Application Publication
(10) Pub. No.: US 2003/0100520 A1
(43) Pub. Date: May 29, 2003

(54) IMMUNOLOGICAL PROCESS AND CONSTRUCTS FOR INCREASING THE HDL CHOLESTEROL CONCENTRATION BY DNA VACCINATION

(76) Inventors: PHILIP NEEDLEMAN, CREVE COEUR, MO (US); KEVIN GLENN, MARYLAND HEIGHTS, MO (US)

Correspondence Address: POLSTER, LIEDER, WOODRUFF & LUCCHESI, 763 SOUTH NEW BALLAS ROAD, ST. LOUIS, MO, 63141-8750, US

(21) Appl. No.: 09/386,591

(22) Filed: Aug. 31, 1999

Related U.S. Application Data
(63) Continuation of application No. 08/934,367, filed on Sep. 19, 1997.
Application No. 08/934,367 is a continuation-in-part of application No. 08/785,997, filed on Jan. 21, 1997.
Application No. 08/934,367 is a continuation-in-part of application No. 08/788,882, filed on Jan. 21, 1997.

(51) Int. Cl. A61K 48/00; C07H 21/04

(52) U.S. Cl. 514/44; 536/23.53; 536/23.2

(57) ABSTRACT

A process for inducing the production of antibodies that bind to cholesteryl ester transfer protein (CETP) is disclosed. That process comprises the steps of: (a) immunizing a mammal with an inoculum containing a recombinant DNA molecule that comprises a DNA sequence that contains (i) a sequence encoding a CETP immunogen that is linked to (ii) a promoter sequence that controls expression of the CETP immunogen, the recombinant DNA molecule being dissolved or dispersed in a vehicle; and (b) maintaining the immunized mammal for a time period sufficient to induce the production of antibodies that bind to CETP, and preferably lessen the transfer of cholesteryl esters from HDL where the blood of the mammal itself contains CETP. Immunogens, inocula, DNA segments, and recombinant DNA molecule vectors useful for carrying out the invention are also disclosed.

### CROSS-REFERENCE TO RELATED APPLICATIONS

This is a continuation-in-part of application Ser. Nos. 08/785,997 and 08/788,882, both filed Jan. 21, 1997.

### TECHNICAL FIELD

The present invention relates to a process for inducing antibodies in a mammal by immunization with DNA that encodes cholesteryl ester transfer protein (CETP) or a portion thereof, and more particularly to an immunological process for ameliorating dyslipoproteinemias in immunized mammals characterized by low HDL/LDL cholesterol ratios by means of those induced antibodies as well as specific DNA constructs for use in that process.

### BACKGROUND OF THE INVENTION

Cholesteryl ester transfer protein (CETP) is an acidic plasma glycoprotein that plays a critical role in establishing high density lipoprotein (HDL), low density lipoprotein (LDL), and very low density lipoprotein (VLDL) cholesterol blood plasma levels and lipid composition in plasma [L. Lagrost, Biochem. Biophys. Acta., 1215:209-236 (1994)]. Several studies, some of which are discussed below, have demonstrated that CETP mediates the transfer of cholesteryl esters (CE) from HDL particles to LDL and VLDL particles, as well as mediating the transfer of triglycerides (TG) from LDL and VLDL to HDL particles. This reciprocal exchange of CE and TG is the primary means of providing CE to LDL and VLDL particles in many mammalian species. CETP thus mediates the balanced exchange of cholesteryl esters (CE) and triglycerides (TG) between pro-atherogenic (LDL and VLDL) and anti-atherogenic (HDL) lipoprotein fractions.

Mammalian species whose blood plasma contains CETP such as humans and other primates, rabbits, and hamsters suffer atherosclerosis and heart disease when exposed to diets rich in cholesterol. Other animal species such as mice, rats and dogs lack plasma CETP (measured as transfer activity) and are not susceptible to dietary cholesterol-induced atherosclerosis.

That CETP contributes to the pathogenesis of atherosclerosis in humans has been strongly supported by transgenic mouse studies [G. Melchior et al., Trends in Card. Med, 5:83-87 (1995)]. For example, transgenic mice having a mini gene of cynomolgus monkey CETP cDNA plus the proximal region of the CETP promoter show dietary cholesterol regulation of CETP levels similar to those seen in humans, hamsters and monkeys. Those transgenic mice expressing high levels of the monkey CETP (levels comparable to human dyslipidemias) exhibit: increased LDL+VLDL cholesterol and apo-B and, decreased HDL cholesterol, LDL-receptor and HMG-CoA reductase mRNA. Atheroma could be induced by high fat diet in transgenic mice with the cynomolgus monkey CETP transgene.

The CETP amino acid residue and nucleotide sequences of mammalian species have been characterized. For example, the human CETP DNA sequence of SEQ ID NO:1 has been determined [D. Drayna et al., Nature, 327:632-634 (1987)]. The rabbit CETP DNA sequence of SEQ ID NO:27 has also been characterized [M. Nagashima et al., J. Lipid Res., 29:1643-1469 (1988); Kotake et al., J. Lipid Res., 37:599-605(1996) and Kotake et al., Biochim. Biophys. Acta., 1347:69-74 (1997)], as has the cynomolgus monkey CETP sequence SEQ ID NO:31 [M. E. Pape et al., Atherosclerosis and Thrombosis, 11:1759-1771 (1991)]. The human CETP protein is 476 amino acid residues long, whereas the rabbit CETP protein is 496 amino acid residues long, and the cynomolgus monkey sequence contains 476 residues.

CETP may be a key factor for the global regulation of atherogenicity of plasma lipoproteins in patients with atherosclerosis or coronary artery disease (CAD). CAD is the number one cause of morbidity and mortality in western society. Patients at increased risk for developing coronary artery disease typically exhibit an enhanced level of CETP activity. It has also been reported that CETP has higher affinity for oxidized LDL than native LDL molecules [L. Lagrost, Biochem. Biophys. Acta., 1215:209-236 (1994)]. High levels of LDL cholesterol (<180 mg/dl) [J. Am. Med. Assoc., 269:3015-3023 (1993) and A. L. Gould et al., Circulation, 91:2274-2282 (1995)]; and low levels of HDL cholesterol (>35 mg/dl) [G. Assman et al., Excerpta Medica, 46-59 (1989) and V. Manninen et al., Circulation, 85:37-45 (1992)] have been reported to be important contributors to the development of atherosclerosis.

Individuals who possess genetic deficiencies of the CETP protein have elevated HDL cholesterol levels. Heterozygotes have HDL levels 15-20 percent above non-affected controls. It has been suggested that there is a 2-3 percent decrease in incidence for CAD for each 1 mg/dl increase in HDL cholesterol after correction for other risk factors [D. J. Grodon et al., Nature, 79:8-15, (1989)].

In an experimental model of CETP deficiency in hamsters, it has been shown that passive transfer of mouse anti-human CETP monoclonal antibodies (1C4) inhibited hamster plasma CETP CE transfer by 70-80 percent at all times up to 24 hours following injection of 500 μg of 1C4 (approximately 3.7 mg/kg body weight). That inhibition of hamster CETP-mediated transfer in vivo increased hamster HDL cholesterol by 33 percent, increased HDL-CE by 31 percent and decreased HDL-TG by 42 percent. These results indicate an example of mammalian (hamster) CETP-mediated CE-TG exchange being disrupted by xenogeneic anti-human CETP monoclonal antibodies, and further demonstrate the use of hamsters as a pre-clinical model for testing CETP inhibition [B. J. Gaynor et al., Atherosclerosis, 110(1):101-109 (1994)].

In another study reported by G. W. Melchior et al., J. Biol. Chem., 270(36):21068-74 (1995) cynomolgus monkey CETP was shown to have two neutral lipid binding sites. A monoclonal antibody to purified cynomolgus monkey CETP identified as CMTP-2 was capable of severely inhibiting triglyceride (TG) transfer, but had a variable effect on cholesteryl ester (CE) transfer.

Thus, when the monoclonal antibody was administered sub-cutaneously to cynomolgus monkeys at a dose that inhibited TG transfer in the plasma by more than 90 percent, there was no detectable effect on the high density lipoprotein cholesterol level, but the HDL-TG levels decreased from 13 to 1 mol/mol of HDL. A Fab antibody fragment had no effect on CE transfer, but completely blocked TG transfer. Another type of inhibitor, 6-chloromercuric cholesterol, severely inhibited CE transfer with minimal inhibition of TG transfer. When both the inhibitory monoclonal antibody and the 6-chloromercuric cholesterol were added to the assay, both CE and TG transfer were inhibited, indicating that the inhibitors did not compete for the same binding site on CETP. This study indicated that in vivo administration of xenogeneic monoclonal antibodies uncoupled CE and TG transfer.

The inhibitory effects of anti-sense RNA on expression of CETP protein were reported using vaccinia virus as an expression system. [M. H. Lee et al., J. Biochem. Mol. Biol., 28(3) :243-248 (1995)]. The cDNA from CETP was inserted into a transfer vector (pSC11) in sense and anti-sense orientations and then used to construct recombinant vaccinia viruses. Decreased expression of the exogenous CETP cDNA in mouse cells was clearly evident in the Northern and Western blot analyses as the dose of anti-sense expression increased. Also, in the CETP assay, the CETP activity was decreased compared to the activity obtained from the cell extracts infected with sense constructs only.

More recently, Sugano et al., J. Biol. Chem., 271(32) :19080-19083 (1996) reported upon the in vivo effects of anti-sense CETP RNA administration to rabbits. In that report, decreases in total cholesterol and CETP activity levels were found 24, 48 and 96 hours following anti-sense CETP administration, as was an increase in plasma HDL cholesterol at 48 hours.

Other methods of inhibition of CETP-mediated transfer are described in the literature. For example, data from Parke-Davis company has shown that infusion of 10 to 20 mpk of the small molecule compound referred to as PD 140195 into rabbits inhibited CETP activity within 30 minutes (measured in an ex vivo assay) [C. Bisgaier et al., Lipids, 811-818 (1994)]. Schering-Plough Company has published on the isolation of Wiedendiol-A and -B from a marine sponge and has shown that this class of compounds to be low μM inhibitors of CETP-mediated CE transfer in vitro [S. Coval et al., Bioorganic & Med. Claim. Lett., 5:605-619 (1995)].

Currently, nicotinic acid and the fibrate drugs are the only small molecule drug therapies that cause significant rises in HDL cholesterol. These drugs are poorly tolerated and must be taken daily. Therapeutic doses of these drugs lead to 15-20 percent increases in HDL cholesterol.

Three mouse monoclonal antibodies to human CETP that recognize a similar epitope on CETP, caused parallel and complete in vitro immunotitration of human plasma CE and triglyceride transfer activities, but only partial inhibition of phospholipid transfer activity [C. B. Hesler et al., J. Biol. Chem., 263(11):5020-5023 (1988)]. Those three monoclonals were originally designated 5C7, 2H4 and 7E1, but in more recent publications of the authors, those monoclonals are referred to as TP2, TP1 and TP3, respectively.

Monoclonal antibody TP2 is directed against an epitope within the last 26 amino acids of CETP (SEQ ID NO:29) [T. L. Swenson et al., J. Biol. Chem., 264:14318-14326 (1989)], and more particularly to an epitope between about positions 465 and 475 of SEQ ID NO:28 [Tall, J. Lipid Res., 34:1255-1274 (1993)] That monoclonal has been shown to block CETP-mediated lipid transfer by limiting access to lipid-binding sites in the carboxy-terminus of CETP.

In an in vivo study using the xenogeneic mouse monoclonal antibodies (TP1) to CETP, rabbits were intravenously injected with TP1, or irrelevant monoclonal antibodies or saline (control), resulting in an initial 70 percent inhibition of CETP-mediated CE transfer activity. Inhibition was 45 percent after 48 hours for the TP1-injected animals. HDL-CE increased in TP1-treated animals and reached levels that doubled over initial and control values at 48 hours. HDL-TG fell reciprocally, but HDL protein did not change, suggesting a CE for TG exchange. VLDL CE-TG ratio also decreased. CETP inhibition delayed the initial clearance of radioactively-tagged HDL, suggesting that CETP plays a quantitative role in HDL-CE catabolism in the rabbit, promoting the exchange of TG for CE, and the clearance of CE from plasma [M. E. Whitlock et al., J. Clin. Invest., 84:129-137 (1989)].

In further animal studies with hamsters, a single sub-cutaneous injection of TP2 monoclonal antibodies in another illustration of passive administration of xenogeneic antibodies decreased CETP-mediated activity by 58 percent, lowered LDL+VLDL cholesterol 32 percent and raised HDL cholesterol 24 percent [G. Evans et al., J. Lipid Res., 35:1634-1645 (1994) and S. Zuckerman et al., Lipids, 30:307-311 (1995)]. The effect of the TP2 monoclonal antibodies on CETP-mediated CE transfer inhibition was evident within 24 hours after injection and was maximized by 4 days. Lipoproteins returned to control levels 14 days after TP2 administration. The shift in the ratio of VLDL+LDL cholesterol to HDL cholesterol levels due to TP2 monoclonal antibody administration was more significant in hypercholesterolemic hamsters.

TP2 also has a higher efficacy in hamsters fed with a western diet enriched in cholesterol. CETP-mediated activity was reportedly increased in these animals 2-fold over chow-fed hamsters.

The preparation of recombinant CETP molecules has been reported by several research groups. For example, in a study reported recently, glutathione S-transferase-human CETP fusion protein (86 kDa) was expressed using vaccinia viral transfer vectors transfected into CV-1 monkey kidney cells. Using a Western blot assay, the fusion protein was identified by polyclonal antibodies against the carboxy-terminal active region of CETP fused with GST. After cleavage of the GST portion of the fusion protein, the purified CETP showed biological activity in a CETP in vitro assay [W. H. Yoon et al., Mol. Cells, 5(2):107-113 (1995)] and M. K. Jang et al., J. Biochem. Mol. Biol., 28(3):216-220 (1995)].

It has also been reported that specific rabbit polyclonal antibodies were produced by immunization with a GST-CETP fusion protein. A full-length CETP cDNA clone isolated from a human heart λgt11 library was used to provide the C-terminal 94 bp of CETP after a full length CETP molecule expressed in E. coli was found to be insoluble. The λgt11 cDNA library was subcloned into pGEX plasmid and a GST-CETP fusion protein was expressed in E. coli. The CETP-GST fusion protein was purified by glutathione-Sepharose-4B affinity chromatography and used as an antigen for the production of rabbit polyclonal antibodies. The antibodies showed good titers, not only against the GST-CETP fusion protein, but also against a mixture of synthetic peptides corresponding in sequence to two 16-mers from the carboxy-terminal region of human CETP. The antibodies were said to be useful as an immunological tool for a CETP assay [N. W. Jeong et al., Mol. Cells, 4(4):529-533 (1994)].

To date there are no published reports on the long-term inhibition of CETP-mediated CE transfer. Passive immunization with the use of xenogeneic antibodies can only be utilized for a short-term period of time because host animals develop antibodies to the xenogeneic immunoglobulin.

One strategy for the generation of a host-induced immune response as compared to xenogeneic antibody use is based on DNA vaccine or “genetic immunization” technology. [W. M. McDonnell et al., N. Engl. J. Med., 334(1): 42045 (1996).] Typically, a DNA vaccine contains a vector that includes one or more genes encoding an antigenic portion of a virus, such as an envelope, surface or a core protein. Host cells take up the DNA vector, express the heterologous gene, and produce the corresponding viral protein inside the cell.

One advantage of this approach is that the viral protein enters and is processed by the cell's major histocompatibility complex (MHC) class I pathway. MHC class I molecules carry peptide fragments to the cell surface where they evoke cell-mediated immunity by stimulating CD⁺8 cytotoxic T cells. Standard vaccine antigens enter cells by phagocytosis or endocytosis and are processed through the MHC class II system, to stimulate antibody responses.

The rapid development of DNA vaccine technology within the past several years was spurred by the report that direct injections of a gene from the influenza A virus could be used to immunize mice against the disease. [J. B. Ulmer et al., Science, 259: 1745-1749 (1993).] Since then, induction of antibodies has been reported for a variety of pathogen-derived proteins including the influenza NP, HA, M1 proteins; HIV Env, Gag, Rev proteins; bovine herpes virus gp; hepatitis B virus surface and core antigens; rabies virus gp, NP, Plasmodium sp. CSP; Leishmania major gp63; Mycobacterium tuberculosis HSP65, Ag85; Hepatitis C virus nucleocapsid protein; Herpes simplex virus gB, gD, ICP27; Papillomavirus L1; Human T-cell leukemia virus type 1 Env; Lymphocytic choriomeningitis virus NP; Bacillus thuringiensis Endotoxin; Mycoplasma pulmonis ND; and Salmonella typhi OmpC porin. [See the references cited in J. B. Ulmer et al., ASM News, 62(9): 476-479 (1996).] Induction of cytotoxic T lymphocytes (CTL), protective immunization, or both, has been reported for each of these examples.

The potential advantages of DNA vaccines, in terms of efficacy and cost, over vaccines prepared by traditional approaches is considered to be as significant as two other major advances that were developed over the past century. The first development pioneered by Louis Pasteur was the use of attenuated and killed forms of microorganisms. The second major development was the use of defined components of whole organisms and the use of purified recombinant proteins. The DNA vaccine approach, has been termed the “third vaccine revolution.” [B. Dixon, Bio/Technology, 13(5): 420 (1995).]

DNA vaccines offer several advantages over other types of vaccines. [J. B. Ulmer et al., ASM News, 62(9): 476-479 (1996).] First, expression of antigen encoded on the vector introduced into host cells leads to production of structurally relevant proteins, which are appropriately modified, and induction of cytotoxic T lymphocytes. Second, DNA vaccines induce CTL responses without resorting to complicated protein formulations or to attenuated live organisms. Attenuated live organisms, such as bacteria, or viruses, also have a greater inherent ability to mutate to more virulent forms. Third, expression of antigens after DNA vaccination can persist, for months in some cases, sufficient to promote the induction of memory immune cells. Still further, DNA vaccines can easily be injected intramuscularly or intradermally in simple aqueous solutions, or coated onto metal particles which are blasted into cells with gene guns, which facilitates administration and their subsequent analysis. [Finan, E. F., et al., Proc Natl Acad. Sci. U.S.A. 90: 11478-11482 (1993)].

The invention described hereinafter provides an autogeneic immunological process for the production of antibodies to CETP and can provide long-term lessening of transfer of cholesteryl esters from HDL particles in mammals whose blood contains CETP by utilization of a DNA vaccine. This process permits the long-term elevation of anti-atherogenic HDL cholesterol concentrations.

### BRIEF SUMMARY OF THE INVENTION

The present invention contemplates an autogeneic immunological process for lessening the transfer of cholesteryl esters from HDL particles and for increasing the HDL cholesterol concentration of a mammal whose blood also contains CETP. A contemplated process is useful in treating human pro-atherogenic dyslipoproteinemias characterized by low HDL/LDL cholesterol ratios. Also contemplated here are isolated and purified DNA that encode a useful immunogen and expression systems for that DNA.

One contemplated process comprises the steps of:

(a) immunizing the mammal to be treated with an inoculum containing a DNA molecule that encodes a CETP immunogen that is an immunogenic polypeptide having a CETP amino acid residue sequence and which DNA molecule is dissolved or dispersed in a vehicle; and

(b) maintaining the immunized mammal for a time period sufficient for the immunizing DNA to express the immunogenic polypeptide to induce the production of antibodies that bind to CETP, and preferably also lessen the transfer of cholesteryl esters from HDL. In one embodiment, the DNA encodes an immunogenic polypeptide that is an intact CETP molecule such as recombinant human or rabbit CETP. In another embodiment, the encoded immunogenic polypeptide is a portion of a CETP molecule that is covalently bonded to an exogenous antigenic carrier as a fusion protein.

In preferred embodiments, the exogenous antigenic carrier is the hepatitis B core protein (HBcAg) or diphtheria toxoid. HBcAg is particularly preferred as an encoded exogenous antigenic carrier, that forms a fusion protein with an immunogenic polypeptide having an amino acid residue sequence of the carboxy-terminal 30 residues of CETP. That more preferred fusion protein constitutes a polypeptide having the amino acid residue sequence of the hepatitis B core antigen from which about 3 to about 53 amino acid residues have been deleted and replaced by the immunogenic polypeptide that more preferably still has a length about equal to the number of amino acid residues deleted from HBcAg. The resulting fusion protein is most preferably expressed as particles having the size of HBcAg particles (about 27 nm).

The present invention has several benefits and advantages. One salient benefit is that a contemplated process can be utilized to lessen the CE transfer from HDL to LDL or VLDL, thereby increasing the concentration of anti-atherogenic HDL cholesterol.

An advantage of the invention is that a contemplated process can have an effect that lasts for months as compared to the short-term effects of the small molecule drugs now available.

Another benefit of a contemplated process is that it utilizes the host mammal's own (autogeneic) immunological system to provide a desired result, thereby obviating problems associated with repeated administration of xenogeneic antibodies that themselves become immunogenic in the host mammal.

Another advantage of some contemplated processes is that their use of well known and accepted exogenous antigenic carriers such as HBcAg, tetanus toxoid, and diphtheria toxoid can boost the host mammal's immunity to those pathogens.

Still further benefits and advantages of the present invention will become apparent to a skilled worker from the disclosure that follows.

### Definitions

The term “recombinant” is used to denote version of a DNA, RNA, or protein molecule altered with respect to the native molecule and resulting from the deletion, substitution, or insertion into the chain, by chemical, enzymatic, or biological means, of a sequence (a whole or partial chain of DNA, RNA, or protein) not originally present in that chain.

The term “recombinant DNA molecule” is used to mean a hybrid DNA sequence comprising at least two nucleotide sequences not normally found together in nature.

The term “polypeptide” is used herein to denote a sequence of about 10 to about 500 peptide-bonded amino acid residues. A whole protein as well as a portion of a protein having the stated minimal length are polypeptides.

The term “fusion protein” is used to denote the expression product of two or more different genes in which the amino acid residue sequences of both genes are expressed peptide-bonded together as a single molecule. It is noted that a fusion protein need not have the full length amino acid residue sequence of any protein, but rather usually contains two or more truncated sequences. The term is therefore somewhat of a misnomer, but is nonetheless well known and used as defined here by those skilled in the art.

The term “fused”, when referring to expression of a fusion protein, is used herein to mean peptide-bonded

The term “whole length CETP” is used to denote the full length CETP molecule (for example 476 amino acid residues long for human CETP or 496 residues long for rabbit CETP) as available in nature or produced as a recombinant protein.

The term “CETP immunogen” is used to denote molecule that is used to induce the production of antibodies that immunoreact with (bind to) CETP.

The terms “immunogenic polypeptide having a CETP amino acid residue sequence” or “immunogenic polypeptide” are used to denote the anti-CETP antibody-inducing portion of a “CETP immunogen”; i.e., that portion of a CETP immunogen to which induced antibodies bind.

The term “exogenous antigenic carrier” or “carrier” is used herein to denote a molecule foreign to the immunized mammal that provides a signal to antibody-producing B cells. Such carriers and their functions are well known in the art. Such a carrier can be a polypeptide having a sequence of as few as about 10 amino acid residues to the length of an intact protein, as well as being a synthetic polymer or oligomer.

The term “inoculum” in its various grammatical forms is used herein to describe a composition containing an amount of CETP immunogen (e.g., DNA encoding a polypeptide conjugate, CETP protein or recombinant protein) sufficient for a described purpose that is dissolved or dispersed in an aqueous, physiologically tolerable diluent.

The term “expression” is used to mean the combination of intracellular processes, including transcription and translation undergone by a structural gene to produce a polypeptide.

The terms “operatively linked” or “operably inserted” are used to mean that two or more DNA sequences are covalently bonded together in correct reading frame.

The term “promoter” is used to mean a recognition site on a DNA sequence or group of DNA sequences that provide an expression control element for a gene and to which RNA polymerase specifically binds and initiates RNA synthesis (transcription) of that gene.

The term “structural gene” is used to mean a DNA sequence that is expressed as a polypeptide; i.e., an amino acid residue sequence.

The term “vector” is used to mean a DNA molecule capable of replication in a cell and/or to which another DNA segment can be operatively linked so as to bring about replication of the attached segment. A plasmid is an exemplary vector.

The term “expression vector” is used to mean a DNA sequence that causes a polypeptide to be expressed in that the DNA sequence contains control elements that regulate expression of structural genes when operatively linked to those genes within a vector.

### DETAILED DESCRIPTION OF THE INVENTION

present invention relates to a process for producing antibodies to CETP. Preferably, the produced antibodies lessen the transfer of cholesteryl esters from HDL, and increase the ratio of HDL cholesterol to LDL cholesterol in the blood of a treated mammal that has CETP in its blood. In humans, that increase in HDL to LDL ratio can lead to an amelioration of dyslipoproteinemias characterized by low HDL/LDL cholesterol ratios. That desired raising of the HDL/LDL cholesterol ratio is accomplished immunologically by antibodies induced in the blood of the treated mammal that recognize and bind to circulating CETP. Also contemplated in this invention are a DNA that encodes an immunogen utilized in the process, an inoculum that utilizes the DNA, and an isolated and purified DNA segment that encodes a contemplated immunogen.

I. The Process

A contemplated process is referred to herein as utilizing “autogeneic” antibodies to denote that the useful antibodies are those induced in the host mammal itself. This autogeneic immunological process is therefore to be distinguished from a xenogeneic process in which antibodies from an animal of one species are administered to an animal of another species as where mouse anti-CETP TP2 or 1C4 monoclonal antibodies have been administered to hamsters or rabbits. A contemplated autogeneic immunological process is also to be distinguished from an allogeneic immunological process such as a passive immunization in which antibodies from one animal are administered to another animal of the same species as where humans receive gamma globulin injections from other humans.

A contemplated process is thus closely analogous to an autoimmune process in which a mammal's own immune system attacks an endogenous or self protein. CETP is an endogenous protein in rabbits, hamsters and primates that are among the mammalian hosts contemplated here. However, inasmuch as the cause of most if not all autoimmune responses is presently unknown and the desired immune response contemplated here is purposefully induced, it is believed appropriate to use a different name for the result obtained here.

One contemplated process produces antibodies to CETP in a mammal. That process comprises the steps of:

(a) immunizing the mammal with an inoculum containing a vehicle in which is dissolved or dispersed a recombinant DNA molecule comprising a DNA sequence that contains (i) a sequence encoding a CETP immunogen linked to (ii) a promoter sequence that controls the expression of the CETP immunogen DNA sequence in the mammal. The encoded CETP immunogen is an immunogenic polypeptide having a CETP amino acid residue sequence. The immunization provides an amount of the recombinant DNA molecule sufficient to be expressed and for the expressed immunogenic polypeptide to induce antibodies to CETP. The immunized mammal is (b) maintained for a time period sufficient for production of antibodies that bind to CETP.

Another aspect of the present invention contemplates a process for lessening the transfer of cholesteryl esters from HDL particles and increasing the concentration of HDL cholesterol in the blood of a mammal whose blood contains cholesteryl ester transfer protein (CETP); i.e., animals that have endogenous plasma CETP measured as a transfer activity. That process comprises the steps of: (a) immunizing that mammal (the host) with an inoculum that contains a DNA-encoded CETP immunogen and linked, controlling promoter sequence dissolved or dispersed in a vehicle. The DNA-encoded CETP immunogen is an immunogenic polypeptide having a CETP amino acid residue sequence. The immunized mammal is (b) maintained for a time period sufficient for the DNA to express the immunogenic polypeptide and for the expressed immunogenic polypeptide to induce the production of antibodies that bind to CETP, and preferably lessen the transfer of cholesteryl esters (CE) from HDL.

A. The CETP-Encoded Immunogen

The immunizing DNA that encodes an immunogenic polypeptide having a CETP amino acid residue sequence of the CETP immunogen can encode a whole CETP molecule such as the human (476 residues) or rabbit (496 residues) proteins whose amino acid residue sequences are provided as SEQ ID NOs:28 and 26, respectively, and whose DNA sequences are provided in SEQ ID NOs:l and 27, respectively. The cDNA (SEQ ID NO:31) and deduced amino acid residue sequence (SEQ ID NO:30) for cynomolgus monkey CETP have also been reported by Pape et al., Atherosclerosis and Thrombosis, 11:1759-1771 (1991). A DNA that encodes a polypeptide of SEQ ID NO:30 or a portion thereof as described below, can also be utilized herein; i.e., the cDNA shown in SEQ ID NO:31 or a portion thereof, respectively.

Where the whole CETP molecule is encoded by the DNA as the immunogenic polypeptide of the CETP immunogen, it is preferred to use the DNA sequence that encodes a CETP sequence from an animal species other than that of the immunized mammal; i.e., the encoded CETP is preferably xenogeneic as to the immunized mammal. When an encoded immunogenic polypeptide is other than an intact CETP molecule, it is preferred to use a DNA that encodes a polypeptide having a length of about 10 to about 30 amino acid residues, and more preferably, a length of about 20 to 30 residues. In this instance, the immunogenic polypeptide is expressed covalently (peptide) bonded to an exogenous antigenic carrier to form the CETP immunogen. The encoded immunogenic polypeptide and antigenic carrier sequences are, of course, linked together in proper reading frame, as is an encoded immunogenic polypeptide linked to the promoter when used without an antigenic carrier.

Exogenous antigenic carrier polypeptide molecules are also well known in the art, as are the amino acid residue and nucleotide sequences of those molecules. Exemplary polypeptide carriers include but are not limited to tetanus toxoid, diphtheria toxoid, thyroglobulin and the hepatitis B core protein (HBcAg).

Thus, the cDNA encoding an exogenous antigenic carrier and that encoding an immunogenic CETP polypeptide are operatively linked to form a single isolated and purified DNA molecule that encodes both the carrier and immunogenic polypeptide. That DNA molecule can then be operatively linked in an appropriate expression vector along with a promoter that controls expression of those two polypeptides as a single fusion protein whose two polypeptide portions are covalently bonded by a peptide bond. Preferably, the carrier is expressed at the amino-terminus of the fusion protein, although a carrier can also be expressed fused at the carboxy-terminus of the immunogenic polypeptide. Exemplary proteins and procedures for their synthesis are discussed hereinafter.

Preferably, where the whole CETP molecule is used as the immunogenic polypeptide, the carrier polypeptide has an amino acid residue sequence that is less than that of a whole protein. That length is preferably about 15 to about 70 amino acid residues.

The hepatitis B nucleocapsid or core protein antigen also referred to as HBcAg is a particularly preferred exogenous antigenic carrier, as will be discussed in greater detail hereinafter. The HBcAg molecule is often used illustratively herein as a carrier.

U.S. Pat. No. 4,818,527, whose disclosures are incorporated by reference, teaches that the region extending from about position 70 through about position 140 from the amino-terminus of HBcAg, whose complete amino acid and cDNA sequences are shown as SEQ ID NOs:38 and 39, respectively, is particularly useful as a T cell independent stimulant as are sequences of about 15 to about 25 residues from that region. The amino acid residue sequences of four of those shorter polypeptides are provided as SEQ ID NOs:40, 41, 42 and 43. The cDNA sequences that encode each of those four polypeptides can be readily obtained from SEQ ID NO:39, and the 3′ end of such a cDNA can be operatively linked to the 5′ end of cDNA that encodes an immunogenic polypeptide, or vice versa, for expression as a fusion protein CETP immunogen.

Thus, in one embodiment, a preferred recombinant DNA molecule (defined previously and discussed hereinafter) encodes a CETP immunogen that is a fusion protein whose amino-terminal portion is a polypeptide having a length of about 15 to about 70 amino acid residues and having the sequence of HBcAg from about position 70 to about position 140 from the HBcAg amino-terminus. The carboxy-terminal portion of that fusion protein has the amino acid residue sequence of a CETP molecule, and the two portions are covalently bonded by a peptide bond. In this embodiment, the CETP molecule can be from the same species as the immunized mammal.

In another preferred embodiment, the immunizing DNA encodes a CETP immunogen that is comprised of an exogenous antigenic carrier to which one or more immunogenic polypeptides having a length of about 10 to about 30 amino acid residues such as those of SEQ ID NOs:2-7 or 50 having a sequence of rabbit CETP, the similar polypeptides of SEQ ID NOs:8-13 or 29 having a sequence of human CETP or the similar polypeptides of SEQ ID NOs:32-37 having a sequence of monkey CETP is covalently bonded. Here, the carrier is preferably an intact protein such as a before-noted tetanus toxoid, diphtheria toxoid, thyroglobulin or HBcAg molecule.

As noted before, a DNA sequence for the CETP molecule or a desired portion thereof can be obtained as described by M. E. Pape et al., Arteriosclerosis and Thrombosis, 11:1759-1771 (1991); N. W. Jeong et al., Mol. Cells, 4(4):529-533 (1994); and D. T. Connolly et al., Biochem. J., 320:39-47 (1996). Oligonucleotides can also be prepared using standard synthetic technology where shorter DNA sequences are desired. Those oligonucleotides can also be linked enzymatically, as with T4 DNA ligase, to form longer molecules.

DNA sequences for exogenous antigenic carrier molecules have also been reported as have methods for expressing those molecules. For example, a DNA sequence that encodes the preferred HBcAg exogenous antigenic carrier is disclosed in U.S. Pat. No. 4,710,463, whose disclosures are incorporated herein by reference, and E. coli-containing plasmids whose DNA encode hepatitis B virus proteins were deposited in the Culture Collection of the National Collection of Industrial Bacteria, Aberdeen Scotland as pBR322-HBV G-L. In addition, DNA encoding HBcAg is disclosed in U.S. Pat. No. 4,942,125 as present in vectors deposited at the American Type Culture Collection (ATCC), 12301 Parklawn Drive, Rockville, Md. 20852-1776 as ATCC No. 39629, No. 39631 and No. 40102.

The use of HBcAg as an exogenous antigenic carrier in a fusion protein is illustrated in Moriarty et al., Vaccines 90, Brown et al. eds., Cold Spring Harbor Laboratory Press, Cold Spring Harbor, N.Y., 225-229 (1990). The authors there reported operatively linking the 3′ end of DNA encoding a 17-mer amino acid residue sequence of the HIV gag protein to the 51 end of DNA encoding HBcAg, and reported that appropriately transfected E. coli expressed a fusion protein having the HIV gag sequence peptide-bonded to the amino-terminus of HBcAg. That expressed fusion protein was present in particulate form and was shown to be an excellent immunogen in mice.

Schödel et al., Vaccines 90, Brown et al. eds., Cold Spring Harbor Laboratory Press, 193-198 Cold Spring Harbor, N.Y. (1990) reported the preparation and successful use of a fusion protein immunogen that contained a polypeptide immunogen having an amino acid residue sequence of hepatitis B Pre-S2 (residues 133-140) that was expressed peptide-bonded to the carboxy-terminus of HBcAg so that the 3′ end of the exogenous carrier (HBcAg) DNA was linked to the 5′ end of the DNA that encoded the Pre-S2 polypeptide immunogen. That expressed fusion protein immunogen was also obtained in particulate form.

Similar techniques can be utilized here using a CETP-encoding recombinant DNA molecule that contains a DNA molecule of SEQ ID NOs:14-19, 20-25 or a corresponding DNA sequence of SEQ ID NO:31 that encodes a CETP immunogenic polypeptide in place of the DNAs used by the Moriarty et al. or the Schödel et al. groups.

In addition, using similar techniques and others well known to workers of ordinary skill in the recombinant DNA art, a DNA molecule that encodes a fusion protein can be prepared that expresses a polypeptide having an HBcAg amino acid residue sequence such as one of those of SEQ ID NOs:40-43 peptide-bonded to the amino-terminus of an intact CETP molecule.

A particularly preferred DNA-encoded CETP immunogen is a fusion protein comprised of an immunogenic polypeptide having a length of 10 to about 30 amino acid residues that is peptide-bonded to both an amino-terminal flanking amino acid residue sequence and a carboxy-terminal flanking sequence, and is sometimes referred to hereinafter HBcAg/CETP/HBcAg. Those flanking sequences are preferably portions from the amino-terminal and carboxy-terminal regions of the HBcAg molecule, as was discussed previously. Thus, in this fusion protein, the exogenous antigenic carrier molecule is encoded to be peptide-bonded to both the amino-terminus and carboxy-terminus of the immunogenic polypeptide.

A preferred encoded polypeptide immunogen (immunogenic polypeptide) has an amino acid residue sequence of SEQ ID NOs:2-7, 8-13, 29, 32-37 or 50. Most preferably, the encoded polypeptide immunogen has an amino acid residue sequence that is bound by (immunoreacts with) the monoclonal antibodies designated TP1, TP2 and TP3 reported by B. Hessler et al., J. Biol. Chem., 263(11):5020-5023 (1988), or that denominated 1C4 by J. Gaynor et al., Atherosclerosis, 110(1):101-109 (1994). Monoclonal antibody TP2 binds to an epitope located between about positions 465 and 475 of human CETP. Tall, J. Lipid Res., 34:1255-1274 (1993), and the citations therein.

A particularly preferred recombinant DNA molecule contains a controlling promoter sequence linked to an encoded polypeptide immunogen sequence whose encoded amino acid residue sequence includes positions 465 through 475 of human CETP or an analogous position of CETP from another source. An encoded polypeptide is exemplified by those of SEQ ID NOs:4, 10, 29 and 34, of which the encoded polypeptides of SEQ ID NOs:10 and 29 are most preferred. The polypeptide of SEQ ID NO:10 is encoded by the DNA of SEQ ID NO:22.

Protein molecules have not only a linear amino acid residue or primary sequence, but also can possess a secondary sequence in which the polypeptide back bone is coiled in an α-helix or folded into a β-sheet, as well as a tertiary sequence in which sequentially distant portions of the molecule are folded to be adjacent to each other. Many linear antigenic/immunogenic polypeptide sequences have been reported in the literature, and such sequences can be readily mimicked by polypeptides having a length of 10 to about 30 amino acid residues. Such relatively short polypeptides typically do not mimic a secondary structure such as an α-helix in aqueous media.

The region of CETP that immunoreacts with monoclonal antibody TP2 is predicted to have an amphipathic helical secondary structure, with the hydrophilic surface bound by the antibody. See Wang et al., J. Biol. Chem., 267(25) :17487-17490 (1992) and A. R. Tall, J. Lipid Res. 34:1255-1274 (1993). A contemplated DNA encodes a CETP immunogen fusion protein having an immunogenic polypeptide flanked at its amino- and carboxy-termini by peptide-bonded regions of HBcAg; i.e., HBcAg/CETP/HBcAg, that is more constrained in its molecular motions than is an immunogenic polypeptide that is bonded at only one terminus. As a consequence, by flanking a before-mentioned particularly preferred immunogenic polypeptide with regions of HBcAg to form a HBcAg/CETP/HBcAg fusion protein, it is believed that the expressed immunogenic polypeptide becomes constrained to have a helical structure much like that present in the native CETP molecule and thereby induce autogeneic antibodies having an antigenic specificity similar to those exhibited by mouse monoclonal antibodies TP1, TP2, TP3 and 1C4 discussed previously.

It is further believed that formation of HBcAg-like particles of an expressed fusion protein HBcAg/CETP/HBcAg immunogen places further conformational constraints upon the immunogenic polypeptide by which the immunogenic polypeptide becomes the primary immunogen with loss of much of the HBcAg immunogenicity, while the T cell-independent antigenic carrier function of HBcAg is retained. See Schödel et al., J. Virol., 66(1):106-114 (1992) for a similar result using a different immunogen.

Although use of the full length HBcAg exogenous antigenic carrier molecule or substantially full length molecule has thus far been discussed, it is noted that about 10 amino-terminal amino acid residues (about 30 base pairs) or about 40 carboxy-terminal amino acid residues (about 120 base pairs) can be deleted from the expressed HBcAg/CETP/HBcAg sequence (encoding DNA) without abrogating function as an exogenous antigenic carrier or assembly into particles. See, for example, Birnbaum et al., J. Virol., 64(7):3319-3330 (1990).

Exemplary preparations of immunogenic fusion proteins having HEcAg as a carrier with various heterologous polypeptide insertions from pathogens as immunogen, and also usage of full length and carboxy-terminal deletions in the HBcAg amino acid residue sequence can be found in the following publications. Schödel et al., J. Exp. Med., 180:1037-1046 (1994); Schödel et al., J. Virol., 66(1):106-114 (1992); Schödel et al., Vaccines 91, Brown et al. eds., Cold Spring Harbor Laboratory Press, Cold Spring Harbor, N.Y., 319-325 (1991); Clarke et al., Vaccines 91, Brown et al. eds, Cold Spring Harbor Laboratory Press, Cold Spring Harbor, N.Y., 313-318 (1991); and Schödel et al., Vaccines 90, Brown et al. eds., Cold Spring Harbor Laboratory Press, Cold Spring Harbor, N.Y., 193-198 (1990).

It is also noted that the human hepatitis virus (HBV), whose core antigen is discussed herein, has two subtypes that are denominated adw and ayw. The core antigens of those two viral subtypes have slightly different DNA and amino acid residue sequences. Although subtype specificity has been noted as to the immunogenicity of the S and PreS regions of HBV, [see, for example, Milich et al. Vaccines 86, Brown et al. eds., Cold Spring Harbor Press, Cold Spring Harbor, N.Y., 377-382 (1986)] either subtype can be used as an exogenous antigenic carrier herein, with subtype ayw being used illustratively herein.

It should also be noted that although a contemplated process has to a great extent been discussed in terms of the polypeptide immunogen that is ultimately expressed and induces autogeneic antibody production to CETP, that immunogenic polypeptide may not be readily discernable in an immunized host mammal. The DNA that encodes such an immunogenic polypeptide may also not be easily identifiable in the mammalian host where no reporter gene such as β-galactosidase is included as a part of the immunizing recombinant DNA molecule, or where an immunizing recombinant DNA molecule, which exists and functions extrachromosomally, is eliminated from an immunized cell at a time subsequent to expression of a CETP immunogen. Antibodies that bind to CETP are, however, present in the host after an appropriate amount of time, and the presence of those antibodies provides evidence that a contemplated process has been carried out in that such CETP-binding antibodies are not known to arise naturally.

B. DNA Molecules and Expression Systems

A contemplated DNA molecule (isolated purified DNA segment) that encodes a CETP immunogen can be referred to as a number of base pairs at a particular location in a plasmid, as a restriction fragment bounded by two restriction endonuclease sites, and as a restriction fragment bounded by two restriction endonuclease sites and containing a number of base pairs. A contemplated DNA can also be defined to have a sequence of a denominated SEQ ID NO, as well as alleles or variants of such genes (described hereinafter) that encode a recited amino acid residue sequence.

A contemplated isolated and purified DNA segment is linear, and as such has a 5′ end and a 3′ end. A contemplated DNA segment can comprise two or more individual DNA segments whose 3′ ends are operatively linked to the 5′ end of another DNA segment where two segments are joined, or whose 3′ end is operatively linked to the 5′ end of another DNA segment whose own 3′ end is operatively linked to the 5′ end of yet another DNA segment, where three individual segments are joined to form a single isolated and purified DNA segment.

In living organisms, the amino acid residue sequence of a protein or polypeptide is directly related via the genetic code to the deoxyribonucleic acid (DNA) sequence of the structural gene that codes for the protein. A structural gene can be defined in terms of the amino acid residue sequence; i.e., protein or polypeptide, for which it codes.

In addition, through the well-known redundancy of the genetic code, additional DNA sequences can be prepared that encode the same amino acid residue sequences, but are different from a recited gene sequence having a particular SEQ ID NO. For example, in vitro mutagenesis as is illustrated hereinafter can be used to change a DNA sequence so that the same residue of an expressed polypeptide is expressed using one or more different codons. In addition, that same technique can be used to change one amino acid residue to another where it is desired to insert or delete specific restriction endonuclease sites. This technique is also illustrated hereinafter.

A DNA sequence that encodes a CETP immunogen of a recited SEQ ID NO but has a DNA sequence different from that of a recited SEQ ID NO is referred to herein as a variant DNA sequence. Such a variant DNA molecule can be readily prepared by in vitro mutagenesis, as is well known.

A DNA segment that encodes a described CETP immunogen can be synthesized by chemical techniques, for example, the phosphotriester method of Matteucci et al., J. Am. Chem. Soc., 103:3185 (1981). Of course, by chemically synthesizing the coding sequence, any desired modifications can be made simply by substituting the appropriate bases for those encoding the native amino acid residue sequence.

However, DNA segments including the specific sequences discussed previously are particularly preferred. Furthermore, a DNA segment that encodes a polypeptide can be obtained from a recombinant DNA molecule (plasmid or other vectors) containing that segment.

A DNA segment that includes a DNA sequence encoding a CETP immunogen can be prepared by excising and operatively linking appropriate restriction fragments from appropriate plasmids or other DNA using well known methods. The DNA molecules useful here that are produced in this manner typically have cohesive termini; i.e., “overhanging” single-stranded portions that extend beyond the double-stranded portion of the molecule. The presence of cohesive termini on the DNA molecules of the present invention is preferred, although molecules having blunt termini are also contemplated.

A recombinant DNA molecule useful herein can be produced by operatively linking a vector to an isolated DNA segment that encodes a CETP immunogen to form a plasmid such as those discussed herein. Particularly preferred recombinant DNA molecules are discussed in detail in the examples, hereafter. Vectors capable of directing the expression of the gene in the immunized mammal are referred to herein as “expression vectors”.

The expression vectors described above contain expression control elements including a promoter. The genes that encode an immunogenic polypeptide or other useful sequence are operatively linked to the expression vector to permit the promoter sequence to direct RNA polymerase binding and expression of the desired polypeptide coding gene.

The choice of which expression vector to which a polypeptide-coding gene is operatively linked depends directly on the functional properties desired, e.g. the location and timing of protein expression, and the host cell to be transformed. These are well known limitations inherent in the art of constructing recombinant DNA molecules. However, a vector useful in practicing the present invention is capable of directing the replication and also the expression of the immunogenic polypeptide-coding gene included in the DNA segment to which it is operatively linked.

A cloning vector is also useful herein for making or increasing the amount of a desired DNA, and can also be used to express an immunogenic polypeptide to assay the synthesized DNA. A cloning vector includes a prokaryotic replicon; i.e., a DNA sequence having the ability to direct autonomous replication and maintenance of the recombinant DNA molecule extrachromosomally in a prokaryotic host cell transformed therewith. Such replicons are well known in the art as are cloning vectors, some of which are discussed below.

Those vectors that include a prokaryotic replicon can also include a prokaryotic promoter region capable of directing the expression of the CETP immunogen gene in a host cell, such as E. coli, transformed therewith. Promoter sequences compatible with bacterial hosts are typically provided in plasmid vectors containing one or more convenient restriction sites for insertion of a DNA segment of the present invention. Such cloning and expression vector plasmids are well known in the art. Typical of such cloning and expression vector plasmids are pUC18, pUC19, pBR322, pProEx1, and pFastBac1 available from Life Technologies, Rockville, Md., and pPL and pKK223-3 available from Pharmacia, Piscataway, N.J. These vectors are utilized in the synthesis of the DNA segments useful herein.

In preferred embodiments, the cloning vector used to express an immunogenic polypeptide-coding gene includes a selection marker that is effective in a host cell, preferably a drug resistance selection marker. One preferred drug resistance marker is the gene whose expression results in kanamycin resistance, whereas ampicillin resistance is another such marker. Again, such selective markers are well known.

A variety of methods has been developed to operatively link DNA to vectors via complementary cohesive termini or blunt ends. For instance, complementary homopolymer tracts can be added to the DNA segment to be inserted and to the vector DNA. The vector and DNA segment are then joined by hydrogen bonding between the complementary homopolymeric tails to form recombinant DNA molecules.

Alternatively, synthetic linkers or adapters containing one or more restriction endonuclease sites can be used to join the DNA segment to the integrating expression vector. The synthetic linkers or adapters are attached to blunt-ended DNA segments by incubating the blunt-ended DNA segments with a large excess of synthetic linker or adapter molecules in the presence of an enzyme that is able to catalyze the ligation of blunt-ended DNA molecules such as bacteriophage T4 DNA ligase.

Thus, the products of the reaction are DNA segments carrying synthetic linker sequences at their ends. These DNA segments are then cleaved with the appropriate restriction endonuclease and ligated into an expression vector that has been cleaved with an enzyme that produces termini compatible with those of the synthetic linker. Synthetic linkers containing a variety of restriction endonuclease sites are commercially available from a number of sources including New England BioLabs, Beverly, Mass. A synthetic adapter molecule typically has sticky end and one blunt end and is not cleaved after ligation.

Although preferred, it is not always feasible to design a DNA molecule whose expressed polypeptide has the exact terminal residues of a polypeptide enumerated in a SEQ ID NO. This is because of the limitations inherent in the use of restriction enzymes, synthetic linkers and adapter molecules used for cutting and joining DNA segments.

As a consequence, an expressed polypeptide can contain a few (e.g., one or two) more, less or different amino acid residues at one or both termini of an enumerated sequence. Such slight changes are well tolerated by a contemplated CETP immunogen, particularly when the substitution is conservative and residues such as Cys and Pro are avoided.

A variety of plasmids can be used as DNA vaccine vectors for expressing a contemplated CETP immunogen in a mammalian host. Such vectors optimally include the following components: a strong eukaryotic promoter, a cloning site for insertion of a gene of interest, a polyadenylation termination [poly(A)] sequence, a prokaryotic origin of replication, and a prokaryotic selectable marker. One such vector, pV1J, contains the cytomegalovirus immediate-early promoter with intron A, a bovine growth hormone polyadenylation termination sequence, and an ampicillin resistance gene. [J. B. Ulmer et al., ASM News, 62(9): 476-479 (1996).] Another useful vector denominated pcDNAI/Amp that is available from Invitrogen, Corp. of San Diego, Calif., as well as plasmids pCMV-SPORT-β-gal and pGreen Lantern-1 available from Life Technologies, Rockville, Md. are discussed in detail hereinafter. Other eukaryotic promoters, poly(A) sites, and selectable markers can be substituted without departing from the utility of the vector, as long as the structural gene inserted downstream from the promoter is expressed in mammalian cells. A variety of general mammalian expression vectors, many of which are commercially available, are suitable for use herein as DNA vaccine vectors.

Plasmid DNA can be prepared by a variety of methods. High quality plasmid DNA can be prepared using CsCl gradients to separate covalently closed circular plasmid DNA from linearized plasmid and chromosomal DNA. Other components from lysed bacterial cells such as proteins, RNA, membranes, and cell wall material are generally well-separated from the plasmid DNA by density gradient centrifugation. Standard protocols for CsCl purification are well known in the art. [See, J. Sambrook et al., Molecular Cloning, 2nd ed., Cold Spring Harbor Press, Cold Spring Harbor N.Y., 8-23 (1989).]

Another method suitable for preparation of high quality plasmid DNA is anion exchange chromatography. Direct comparisons of DNA prepared by anion exchange chromatography and DNA banded twice on CsCl gradients reportedly showed no difference in the efficiency of direct gene transfer and genetic immunization for reporter plasmids. [H. L. Davis et al., Biotechniques, 21: 99-99 (1996).] DNA banded once on CsCl gradients was reported to contain less double-stranded closed circular DNA and more RNA contamination than DNA banded twice on these gradients. Endotoxin levels were said to greater, however, for DNA purified by anion exchange chromatography than by CsCl purification. [H. L. Davis et al., Biotechniques, 21: 99-99 (1996).]

A preparative method of purification of supercoiled plasmid DNA for therapeutic applications can also be used [A. P. Green et al., Biopharm, 10(5): 52-62, (1997)]. This method uses ion-pairing reversed phase column chromatography to prepare plasmid DNA free from contaminating DNA, RNA, protein, and endotoxin.

General methods and procedures for characterizing bulk plasmid DNA vectors suitable for use in human gene therapy have been described [M. Marquet et al., BioPharm, 10(5): 42-50 (1997)]. Methods to characterize a master cell bank, by testing plasmid identity, plasmid yield, plasmid stability, and methods to verify the genotype of the host strain and to measure host strain viability and microbial contamination were described. Methods to characterize the bulk product in terms of sterility and for the presence of pyrogens were also described.

Endotoxin is the lipopolysaccharide component of the cell wall of gram-negative bacteria that is released when cells are lysed for recovery of plasmid DNA. Endotoxin can have cytotoxic effects on mammalian cells in vitro or in vivo. [I. P. Wicks et al., Hum. Gene Ther., 6: 317-323 (1995)]. Endotoxin levels can be measured by a chromogenic limulus amoebocyte clotting assay [SCL-100 kit; BioWhittacker, Walkerville, Md.; T. Mossman, J. Immunol. Meth., 65: 55-63 (1983)]. Methods for purification of endotoxin-free plasmid DNA prepared by anion exchange chromatography are now available. [J. Schorr et al., Gene Ther., 1: S7 (1994)]. This method clears the bacterial lysate with a special endotoxin removal buffer before the DNA is purified from lysate on an anion exchange column.

A plasmid or other vector DNA that encodes a contemplated CETP immunogen and contains a promotor sequence as discussed before that controls expression of that immunogen-encoding DNA sequence in proper reading frame comprises a recombinant DNA molecule utilized in immunizing a host mammal. That recombinant DNA molecule dissolved or dispersed in a vehicle comprises an inoculum that is used to immunize the mammal and is discussed further hereinbelow.

C. Inocula

A DNA vaccine vector encoding a CETP immunogen in proper reading frame (a recombinant DNA molecule) and containing a promoter sequence that controls expression of the immunogenic polypeptide is dissolved or dispersed in a pharmaceutically-acceptable vehicle composition that is preferably aqueous to form an inoculum that when used to immunize a mammal induces the production of antibodies that immunoreact with (bind to) CETP. When that recombinant DNA molecule is administered in an effective amount to a mammal whose blood contains CETP those antibodies preferably also lessen the transfer of cholesteryl esters from HDL particles.

An effective recombinant DNA molecule dosage is typically about 0.05 μg/kg to about 50 mg/kg, usually about 0.005 mg/kg to about 5 mg/kg. Methods of determining the effective systemic dose, which dose can vary depending on the activity of the polypeptide encoded by the DNA can be determined in a manner apparent to those of skill in the art. U.S. Pat. No. 5,580,859, whose disclosure is herein incorporated by reference, discloses several methods of determining an effective dose of a DNA vaccine.

The term “unit dose” as it pertains to an inoculum of the present invention refers to physically discrete units suitable as unitary dosages for animals, each unit containing a predetermined quantity of active material calculated to individually or collectively produce the desired immunogenic effect in association with the required diluent; i.e., carrier, or vehicle.

Inocula are typically prepared from a recombinant DNA molecule encoding a CETP immunogen by dispersing the DNA in a liquid physiologically tolerable (acceptable) diluent vehicle such as water, or phosphate-buffered saline (PBS), or the like to form an aqueous composition. The amount of DNA-encoding CETP immunogen utilized in each immunization can vary widely, and is referred to as an effective amount. Such an effective amount is sufficient to induce antibodies to CETP that bind to CETP, and preferably lessen the transfer of cholesteryl esters from HDL particles, and also increase the HDL/LDL ratio in the immunized mammal's blood. Exemplary effective amounts of a DNA-encoded CETP immunogen are about 0.005 mg/kg to about 5 mg/kg, depending inter alia, upon the sequence of the encoded CETP immunogen, the mammal immunized, and the presence of salts, stabilizers, and cell penetration agents in the inoculum. An exemplary unit does can constitute about 10 μl to about 1 ml per site of administration (immunization), with a recombinant DNA molecule concentration of about 0.05 μg/ml to about 20 mg/ml, and preferably about 0.1 μg/ml to about 100 μg/ml. Thus, a single unit dose or a plurality of unit doses can be used to provide an effective amount of expressed CETP immunogen. Those skilled in the art know that appropriate concentrations or amounts can be readily determined.

An inoculum is typically formulated for parenteral administration. Exemplary immunizations are carried out sub-cutaneously (s.c.) intramuscularly (i.m.) or intra-dermally (i.d.). U.S. Pat. No. 5,580,859, discloses several formulations suitable for use with a DNA vaccine. These include inocula composed of recombinant DNA molecules dissolved or dispersed in an aqueous vehicle containing salts, diluents, stabilizers, and cell penetration agents.

Typical formulations include recombinant DNA molecules suspended in phosphate buffered saline or isotonic sucrose. DNA can also be complexed with cell penetration agents such as liposomes, which facilitate uptake of the DNA into cells. One example of a liposome which has been used in DNA vaccine formulations is Lipofectin™, available commercially from Life Technologies, Rockville, Md. Uptake and expression are often also significantly enhanced if DNA is administered in conjunction with the facilitating agent bupivacaine-HCl (an anesthetic) [Coney, L., et al., Vaccine, 12: 1545-1550 (1994)]. Those skilled in the art will recognize that other similar cell penetration and facilitating agents can be used for these purposes and can that appropriate concentrations or amounts of these agents can be readily determined.

Once immunized, the mammal is maintained for a period of time sufficient for the encoded CETP immunogen to be expressed and then for the expressed CETP immunogen to induce the production of antibodies that bind to CETP and lessen the transfer of cholesteryl esters from HDL particles. This maintenance time typically lasts for a period of about three to about eight weeks, and can include a booster, second immunizing administration of the inoculum.

The production of antibodies that bind to CETP is readily ascertained by obtaining a plasma or serum sample from the immunized mammal and assaying the antibodies therein for their ability to bind to CETP as an antigen in an ELISA assay as described hereinafter, or by another immunoassay such as a Western blot as is well known in the art.

The lessening of transfer of cholesteryl esters from HDL can be assayed by one or more of several techniques. In one assay, the rate of transfer is measured by use of a [³H]-cholesteryl ester ([³H]CE) from HDL to LDL following the differential precipitation assay reported by Glenn et al., Methods in Enzymology, 263:339-350 (1996). Briefly, in a volume of 200 μl, CETP, [³H]CE-labeled HDL, LDL, and TES assay buffer (50 mM Tris, pH 7.4; 150 mM NaCl; 2 mM EDTA; 1% bovine serum albumin) are incubated for 2 hours at 37° C. in 96-well filter plates. LDL is then differentially precipitated by the addition of 50 μl of 1% (w/v) dextran sulfate/0.5 M MgCl₂. After filtration, the radioactivity present in the precipitated LDL is measured by liquid scintillation counting. Correction for non-specific transfer or precipitation is made by including samples that did not contain CETP. The rate of [³H]CE transfer is determined in the linear range of the assay with respect to time and CETP concentration. For studies in which antibodies are included in the assay, the order of addition into sample wells is: buffer, [³H]CE-labeled HDL, LDL, antibodies, CETP.

CETP activity can also be measured using two methods that do not involve differential precipitation. In the first assay, the incubation conditions are identical to those described above, but separation of LDL acceptor particles from [³H]CE-labeled HDL donor particles is accomplished by size exclusion chromatography on tandem columns of Superose™ 6 (Sigma Chemical Co.), followed by liquid scintillation counting of fractions to determine the amount of [³H]CE associated with LDL and HDL. The amount of transfer measured by this method is typically in excellent agreement with the precipitation assay.

Another assay for CETP activity measures the rate of CETP-mediated transfer of the fluorescent analog NBD-cholesteryl linoleate (NBD-CE) from an egg phosphatidyl choline emulsion to VLDL. This assay takes advantage of the fact that NBD-CE is self-quenched when in the emulsion, and becomes fluorescent when transferred to VLDL. The assay is carried out according to the manufacturer's instructions (Diagnescent Technologies Inc., Yonkers, N.Y.). Fluorescence measurements can be taken using a standard machine such as an SLM 8000C spectrophotofluorometer (Milton Roy Co., Rochester, N.Y.) using 465 nm and 535 nm for excitation and emission wavelengths, respectively.

It is particularly contemplated once the desired antibodies are induced in the mammal that the immunization step be repeated at intervals of about 3 to about 6 months until the HDL cholesterol value in the blood of the mammal is increased by about 10 percent or more relative to the HDL cholesterol value for the mammal prior to the first immunization step. Preferably, the HDL cholesterol value is increased by about 25 percent. The mammal is thereafter preferably maintained at that increased HDL cholesterol level by periodic booster immunizations administered at intervals of about 6 to about 18 months. The increase in HDL cholesterol can be measured by any reliable assay, many of which are well known in the art, and one of which is described hereinafter.

It is noted that the before-described anti-CETP antibodies so induced can be isolated from the blood of the host mammal using well known techniques, and then reconstituted into a second inoculum for passive immunization as is also well known. Similar techniques are used for gamma-globulin immunizations of humans. For example, antiserum from one or a number of immunized hosts can be precipitated in aqueous ammonium sulfate (typically at 40-50 percent of saturation), and the precipitated antibodies purified chromatographically as by use of affinity chromatography in which CETP or an immunogenic polypeptide portion thereof is utilized as the antigen immobilized on the chromatographic column.

### BEST MODE FOR CARRYING OUT THE INVENTION

Comparative Examples 1 and 2, below, illustrate results obtained using a CETP polypeptide immunogen prepared exogenously to the immunized mammal.

### Comparative Example 1

### Immunization of Rabbits With Rabbit CETP-Peptides

There is a 88 percent homology between rabbit and human CETP at the amino acid residue level. Rabbits express high levels of CETP in their blood and were chosen as a model for illustrating production of autogeneic anti-CETP antibodies.

The six rabbit CETP polypeptides of SEQ ID NO:2-7 were selected for this study and were prepared by standard solid phase synthesis procedures discussed below. To enhance the anti-polypeptide-specific antibody responses, two separate immunization strategies were used with the above six rabbit CETP-polypeptides.

A. Immunization Strategy 1 (MAP conjugates)

Rabbit polypeptides were synthesized as multiple antigenic peptide (MAP) constructs [D. N. Posnett et al., J. Biol Chem., 263:1719-1725 (1988)]. Those polypeptides were separately covalently bonded to “oligolysine core” molecules that were themselves covalently attached to resin particles [S. Butz et al., Pep. Res. 1:20-223 (1994)].

The substitution of the starting resin particles was 0.37 μm sites/mg resin that provided approximately 500 μg of immunogenic polypeptide per 1.1 mg resin. For the preparation of the CETP immunogen for immunization, 3.0 mg of dry resin were weighed out and hydrated in 1.3 ml sterile phosphate-buffered saline (PBS; pH 7.4) to which 1.3 ml Freund's complete adjuvant (CFA; Sigma Chemical Co., St. Louis, Mo., F-5881) were added as adjuvant. The CETP immunogen and adjuvant were emulsified by a female-female luer lock syringe adapter connected to two 3 ml syringes. Each final emulsion was divided into 1.0 ml aliquots for injection (1 ml/rabbit), with one immunogen used per rabbit. Pre-immune rabbit serum was collected before immunization and stored at −70° C. until immunoassay. On day 1, New Zealand white rabbits were separately immunized with respective immunogens by sub-cutaneous (s.c.) route on the back of the rabbit using 10 injection sites.

Three weeks later (on Day 22), the rabbits were boosted using similar procedures, but this time CETP immunogens were emulsified in Freund's incomplete adjuvant (IFA; Sigma). The resin-bonded CETP immunogen was weighed out as before and hydrated with sterile PBS the day before the booster immunization. The resulting CETP immunogen suspension was sonicated with a microtip at maximum setting for 5 minutes and left overnight (about 18 hours) at 4° C. Before mixing the hydrated CETP immunogen suspension with IFA, the suspension was warmed to room temperature just before the booster immunization, added to 1.5 ml IFA, and emulsified as described above to form an inoculum in which the CETP immunogen was dispersed. Rabbits were immunized each with 1 ml of emulsion in at least 10 injection sites S.C.

The first post-immune serum was collected 2 weeks after the second immunization from each animal. All the anti-sera samples were stored in −70° C. until ELISA was done.

Using this MAP strategy, polypeptides of SEQ ID NOs:2 and 7 were moderately immunogenic in rabbits and resulted in maximum autogeneic antibody titers of 1:1000 and 1:300, respectively. The titers represent the dilution of the sera that gave a half maximal absorbance on ELISA plates coated with the respective polypeptides. Sera were pooled from two rabbits, and the above titers represent the mean value. Only anti-sera to SEQ ID NO:7 cross-reacted with recombinant human CETP. The reactivity of these anti-sera with rabbit CETP is under evaluation using various immunological assays. Anti-polypeptide-specific IgG has been purified from the post-immune sera and its inhibitory property on human recombinant CETP is being assayed.

B. Immunization Strategy 2 (Purified Protein Derivative Conjugates)

Five of the above six rabbit CETP-polypeptide immunogens (SEQ ID NOs:2, 3, 4, 6 and 7) were coupled to tuberculin purified protein derivative (PPD) according to the teachings of P. J. Lachmann et al., in 1986 Synthetic Peptides As Antigens, (Ciba Foundation Symposium 119), 25-40 (1986) and P. Dawson et al., J. Bio. Chem., 264:16798-16803 (1989) to form a conjugate. The tuberculin PPD (Statens Serum Inst., Copenhagen, Denmark) was used as an exogenous antigenic carrier to enhance the immunogenicity of rabbit CETP-derived polypeptides. The polypeptide-PPD conjugate in PBS was emulsified with CFA as described for immunization strategy 1. One ml of 0.5 mg/ml polypeptide conjugated to PPD was emulsified with approximately 1 ml CFA. A second 1 ml PPD-conjugate was frozen for next booster immunization.

On Day 1, rabbits were immunized with 1 ml of final emulsion in at least 10 sites sub-cutaneously on back of the rabbit. The polypeptide-PPD CETP polypeptide immunogen dose contained 0.25 mg of polypeptide per rabbit. Three weeks later (on Day 21), the rabbits were given the booster immunization dose with the remaining 1 ml conjugate thawed and emulsified with IFA, as discussed before. Two weeks following the second immunization rabbits were bled to collect post-immune sera.

The PPD conjugation strategy resulted in antibodies to the immunogenic polypeptides of SEQ ID NOs:2 and 6, with antibody titers of 1:3200 and 1:400 respectively. The titers represent the dilution of the sera that gave a half maximal absorbance on peptide coated ELISA plates. Sera were pooled from two rabbits and represent the mean value. Only the antibodies to the immunogenic polypeptide of SEQ ID NO:2 cross-reacted with recombinant human CETP. These results were unexpectedly good inasmuch as P. J. Lachmann et al., supra, obtained substantially no anti-polypeptide antibodies in BCG-naive hosts as were these rabbits. Anti-PPD antibodies were detected in all groups of rabbits as expected.

Using ELISA, the anti-immunogenic polypeptide sera are being used to evaluate their immuno-reactivity with natural rabbit CETP. Because the polypeptides of SEQ ID NOs:2, 6 and 7 were immunogenic and the two anti-polypeptide antibodies against SEQ ID NOs:2 and 7 immunologically cross-reacted with recombinant human CETP, the respective rabbits were further boosted with a third immunization dose either with the MAP constructs or PPD constructs emulsified with IFA.

### Example 2

### Immunization of Outbred Rabbits With CETP-Based Antigen

This study utilized 30 New Zealand white rabbits in three groups with 10 rabbits per group. Three immunogens were utilized in this study: (1) Recombinant human CETP, (2) the carboxy-terminal 26 amino acid residues of rabbit CETP (SEQ ID NO:50), and (3) a control immunogen whose amino acid residue sequence was unrelated to that of CETP.

Pre-immune sera were collected before immunization with the respective immunogens. The purpose of this study was to illustrate that the above CETP immunogens would induce anti-CETP-specific (autogeneic anti-CETP) antibodies in rabbits, and that the autogeneic antibodies generated against CETP bind to (immunoreact with) the endogenous rabbit CETP, and thus lessen the transfer of cholesteryl esters from HDL particles and raise the level of HDL in the hosts.

The above immunogens were emulsified in CFA. Each rabbit received 500 μg of one of the immunogens emulsified in CFA immunized by sub-cutaneous route. Seven weeks later the first bleed post-immune sera were collected.

ELISA was employed to titrate the antibodies. ELISA plates were coated (40 ng/well) with the recombinant human CETP.

The rabbits immunized with recombinant human CETP exhibited a primary immune response against human CETP. All the ten rabbits responded well to the recombinant human CETP (rhCETP). The specific IgG antibody titer was >1:1000. However, the group of 10 rabbits immunized with the rabbit CETP carboxy-terminal polypeptide-thyroglobulin conjugate (CETP-TH) did not exhibit a primary antibody response. The control rabbit sera had no detectable levels of anti-CETP antibodies. The rabbits were boosted with each respective antigen to further study immunogenicity.

The results of this study on the elevation of HDL particle concentration in the blood (plasma) of the host mammals (mean±.S.D.) are shown in Table 1, below, for those first-immune sera.

TABLE 1

HDL Levels In Immunized
Animals (mg/dl)

| | Avg.³ | | |
|---|---|---|---|
| Immunogen | HDL | S.D.⁴ | P⁵ |
| Control | 23.89 | 3.92 | — |
| rhCETP¹ | 26.59 | 4.41 | 0.17 |
| CETP-TH² | 26.14 | 6.93 | 0.38 |

¹rhCETP = Recombinant human CETP.
²CETP-TH = C-terminal 26 rabbit CETP amino acid residues conjugated to thyroglobulin.
³Avg. HDL = Average HDL concentration after immunization or mock immunization for the control.
⁴S.D. = Standard deviation.
⁵P = p value from a Student's T test analysis.

As can be seen from those results, an increase in HDL particle concentration was found after administration of each of the CETP immunogens. There was a relatively large scatter in the data. Nevertheless, an approximately 10 percent increase in the HDL cholesterol level was observed with each CETP immunogen as compared with the control, with the recombinant human CETP immunogen providing its increase at a confidence level of greater than 80 percent (p=0.17) using a Student's T test to analyze the results.

### Example 3

### Construction of E. coli Expression Vectors Encoding HBcAg/CETP/HBcAg Fusion Proteins

A. PCR amplification of HBcAg

Plasmid pFS14, a derivative of expression vector pKK223 (Pharmacia), encodes HEcAg (subtype ayw) [Schödel et al., Infect. Immun. 57:1347 (1989)]. PCR primer A, below, is designed to amplify the 5′ end of HBcAg and place an NcoI (C′C.ATG,G) site in the correct reading frame at the natural ATG start codon. In each of the sequences shown hereinafter, only the coding strand is shown, and bases removed after cleavage by restriction enzymes are shown in lower case.

| Primer A: | |
|---|---|
| 5′ gatccCATGGACATCGACCCTTATAAAGAATTTG | SEQ ID NO:44 |
| G 3′ | |

Primer Z, below, is designed to amplify the 3′ end of HBcAg and place a TAA stop codon and a HindIII (A′AGCT,T) site following amino acid 183 (Cys).

| | |
|---|---|
| Primer Z: | |
| 5′ gatcaAGCTTTTAACATTGAGATTCCCGAGATTG | SEQ ID NO:45 |
| AGATCTTCTG 3′ | |

A DNA fragment encoding the full-length HBcAg with modified 5′ and 3′ ends is amplified using plasmid pFS14 DNA as a template in the presence of primer A and primer Z under the standard polymerase chain reaction conditions recommended by the manufacturer of the GeneAmp PCR reagent kit (Perkin Elmer Cetus, Norwalk, Conn.).

The amplified DNA is then cleaved with NcoI and HindIII, and fractionated by size on an agarose gel. Full-length HBcAg DNA is purified from a gel slice using a QIAQUICK™ gel extraction kit (QIAGEN, Chatsworth, Calif.).

B. Insertion of HBcAg into pProEx1

pProEx1, an E. coli expression vector (Life Technologies, Rockville, Md.), is also cleaved with NcoI and HindIII and gel-purified. The amplified DNA and pProEx1 DNAs are ligated under standard conditions using T4 DNA ligase and transformed into chemically-competent E. coli DH10B cells (Life Technologies) using protocols supplied by the vendor to form plasmid ProEx1-AZ. The transformation mixture is spread on LB agar plates containing 100 μg/ml ampicillin and incubated overnight at (about 18 hours) at 37° C. Colonies harboring ampicillin-resistant plasmids are purified by restreaking on fresh LB agar plates containing ampicillin, and minipreps of plasmid DNA are prepared using WIZARD™ 373 DNA purification kits (Promega, Inc., Madison, Wis.). Plasmids containing the HBcAg fragment inserted into the NcoI and HindIII sites of pProEx1 are characterized by restriction mapping and sequence analysis across the inserted region.

Plasmid pProEx1-AZ is then modified to insert a polylinker between the nucleotides that encode amino acid residues 70-75 of HBcAg.

Primer B is designed to insert an XhoI site (C′TCGA) and an EcoRI site (G′AATT,C) site following position 206 of SEQ ID NO:39. Primer Y is designed to insert an EcoRI site (G′AATT,C) site followed by a SpeI site (A′CTAG,T) before position 226 of SEQ ID NO:39.

| | |
|---|---|
| Primer Y: | |
| 5′ gatcgAATTCACTAGTTGGAAGATCCAQCGTCTA | SEQ ID NO:46 |
| GAGACCTAGTAG 3′ | |
| Primer B: | |
| 5′ gatcgAATTCCTCGAGCTAGAGTCATTAGTTCCC | SEQ ID NO:47 |
| CCCAGCA 3′ | |

Plasmid pProEx1-AZ is then used as a template with primers A and B to amplify a segment of DNA (designated HBcAg-AB) encoding amino acid residues 1-69 of HBcAg to generate a fragment that contains an NcoI site at its 5′ end and an XhoI and a EcoRI site at its 3′ end. The same plasmid is also used with primers Y and Z to amplify a segment of DNA (designated HBcAg-YZ) encoding amino acid residues 76-183 of HBcAg to generate a fragment that contains EcoRI and SpeI sites at its 5′ end and a HindIII site at its 3′ end.

The PCR product from the reaction designed to produce plasmid HBcAg-AB is cleaved with NcoI and EcoRI and purified after agarose gel electrophoresis. The PCR product from a second reaction designed to produce plasmid HBcAg-YZ is cleaved with EcoRI and HindIII and purified after agarose gel electrophoresis. The two gel-purified fragments are then ligated in a triple ligation reaction to plasmid pProEx1 that had been treated with NcoI and HindIII and purified after agarose gel electrophoresis. The desired ligated plasmid, pProEx1-AB-YZ, is obtained by screening ampicillin-resistant colonies for plasmids that have the predicted structure by restriction analysis, and is confirmed by DNA sequencing across the whole HBcAg region, particularly the A, BY, and Z junctions.

C. Cloning Of CETP Segment Encoding SEQ ID NO:29

A stably transformed CHO cell line transfected with human CETP cDNA [Wang et al., J. Biol. Chem., 270:612-618 (1995); Wang et al., J. Biol. Chem., 267:17487-17490 (1992)] provides CETP cDNA that is used as a template to amplify a segment (nucleotides 1346 to 1431) of the CETP coding sequence (SEQ ID NO: 1) that encodes the human peptide (SEQ ID NO:29; ArgAspGlyPheLeuLeuLeuGlnMetAspPheGlyPheProGluHisLeu LeuValAspPheLeuGlnSerLeuSer) that is bound by the antibody TP2; T. L. Swenson et al., J. Biol. Chem., 264:14318-14326 (1989).

Primer C, below, is designed to amplify a region from just upstream from the natural XhoI site at position 1346. Primer X, below, is designed to amplify a region at the 3′ end of the CETP gene, removing the TAG codon and replacing it with an Eco47III site (AGC|GCT) followed by an EcoRI site (G′AATT,C).

| | |
|---|---|
| Primer C: | |
| 5′ GATTATCACTCGAGATGGCTTCCTGCTGCTGCAG | SEQ ID NO:48 |
| 3′ | |
| Primer X: | |
| 5′ gatcgAATTCAGCGCTCAAGCTCTGGAGGAAAT | SEQ ID NO:49 |
| CCACCAG 3′ | |

The CETP cDNA is then used as a template with primers C and X to amplify a segment of DNA (designated pCETP-CX) encoding amino acid residues 461-476 of CETP, that contains an XhoI site near its 5′ end and an Eco47III and EcoRI site at its 3′ end. This segment, CETP-CX, is then cleaved with XhoI and EcoR47III, and gel-purified. Plasmid pProEx1-AB-YZ is digested with SpeI and treated with T4 DNA polymerase to remove the 4-base 5′ overhangs and generate blunt ends. [See, J. Sambrook et al., Molecular Cloning, 2nd ed., Cold Spring Harbor Press, Cold Spring Harbor N.Y., 8-23 (1989).]

The resulting plasmid is then treated with XhoI, gel-purified, and ligated to the segment CETP-CX that has an XhoI site at one end and a blunt end resulting from cleavage with Eco47III at the other end. The resulting plasmid, designated pProEx1-ABC-XYZ, is characterized by restriction analysis and by sequencing to confirm that it contains sequences encoding amino acid residues 461-476 of CETP replacing sequences that encoded amino acid residues 70-75 of HBcAg in the vector pProEx1-AZ.

### Example 4

### Expression of HBcAg/CETP/HbcAg Fusion Proteins In E. coli

The pProEx1 vector is designed for the expression of foreign proteins in E. coli. This vector contains a gene conferring resistance to ampicillin and a pBR322 origin of replication for propagation in E. coli. It also has a multiple cloning site flanked by a 6 histidine sequence (6×His) and the recognition sequence for rTEV protease. This site allows for the removal of the 6×His tag from a fusion protein after purification. The vector also has a Trc promoter and lacI^{q} gene permitting inducible gene expression with isopropyl-β-D-thiogalactopyranoside (IPTG). A procaryotic ribosomal binding site is located upstream from the start of translation of the 6×His tag. A unique NcoI site is located at the first codon of the 6×His tag. Plasmids pProEx1 and a control plasmid, pProEx1-CAT, are obtained from Life Technologies.

E. coli DH10B strains individually harboring pProEx1, pProEx1-CAT, pProEx1-AZ, or pProEx1-ABC-XYZ are cultured overnight (about 18 hours), and used as inocula for cultures that are induced with IPTG under conditions recommended by the vendor. Cultures harboring plasmid pProEx1-AZ produce HBcAg and those harboring pProEx1-ABC-XYZ produce the desired HBcAg/CETP/HBcAg fusion protein as particles. These proteins lack the 6×His tag present in the original pProEx1 vector because the HBcAg sequences are inserted at the NcoI site at the beginning of the 6×tag. Cultures harboring pProEx1-CAT produce a protein that migrates on SDS-PAGE gels as expected for a His tagged CAT fusion protein.

### Example 5

### Expression of HBcAg/CETP/HBcAg Fusion Proteins In Baculovirus-Infected Insect Cells

Baculovirus-infected insect cells have been shown to express a wide variety of recombinant proteins (V. A. Luckow, Insect Cell Expression Technology, pp. 30 183-218, in Protein Engineering: Principles and Practice, J. L. Cleland et al. eds., Wiley-Liss, Inc, 1996). Heterologous genes placed under the control of the polyhedrin promoter of the Autographa californica nuclear polyhedrosis virus (AcNPV) are often expressed at high levels during the late stages of infection. In most cases, the recombinant proteins are appropriately processed and are functionally similar to their authentic counterparts.

Recombinant baculoviruses containing the chimeric HBcAg/CETP/HBcAg gene are constructed using the baculovirus shuttle vector system (Luckow et al., J. Virol., 67:4566-4579, 1993) sold commercially as the Bac-To-Bac™ baculovirus expression system (Life Technologies).

Briefly, pProEx1-ABC-XYZ is digested with NcoI, treated with Klenow enzyme to fill in the ends, and digested with HindIII to release the entire fragment encoding the HBcAg/CETP/HBcAg fusion protein. This fragment is inserted into a baculovirus donor plasmid, pFastBac1, that is digested with BamHI, treated with Klenow enzyme, and digested with HindIII. The resulting plasmid has the sequences encoding the hybrid HBcAg/CETP/HBcAg gene inserted downstream from the polyhedrin promoter of AcNPV. The mini-Tn7 segment containing the polyhedrin/HBcAg/CETP/HBcAg expression cassette is then transposed to a baculovirus shuttle vector propagated in E. coli and colonies harboring composite (recombinant) vectors are identified by their color and an altered drug resistance patterns. Miniprep DNAs are prepared and transfected into cultured Spodoptera frugiperda (fall armyworm) Sf9 cells.

Stocks of recombinant viruses are prepared and expression of the recombinant protein is monitored by standard protocols (O'Reilly et al., Baculovirus Expression Vectors: A Laboratory Manual, W. H. Freeman and Company, New York, 1992; King, L. A., and Possee, R. D. The Baculovirus Expression System: A Laboratory Guide, Chapman & Hall, London, 1992).

### Example 6

### Expression of HBcAg/CETP/HBcAg Fusion Proteins In Mammalian Cells

The HBcAg/CETP/HBcAg fusion protein is expressed in mammalian cell culture using the BHK/VP16 expression system (Hippenmeyer et al., Bio/Technology, 11:1037-1041, 1993). Briefly, the HbcAg/CETP/HbcAg-encoding sequence of the NcoI-HindIII fragment from plasmid pProEx1-ABC-XYZ of Example 3 is isolated by gel electrophoresis and purified as before. The fragment is treated with Klenow polymerase and all four nucleotide triphosphates to make the 5′ overhanging ends blunt.

The mammalian expression vector pMON3327 contains the SV40 polyadenylation signal sequence in the BamHI site of plasmid pUC18, and is used as the basis for further plasmid construction. Ligation of the IE175 promoter of herpes simplex virus (HSV-1) upstream of the SV40 polyadenylation signal sequence in vector pMON3327 provides mammalian expression vector pMON3360B. The IE175 promoter is responsive to the HSV-1 VP-16 transactivator.

Expression vector pMON3360B is digested with BamHI and the 5′ overhanging ends at the unique BamHI site are filled in using Klenow polymerase. The vector sequences and the HBcAg/CETP/HBcAg sequences are ligated overnight (about 18 hours) at 15° C. using T4 DNA ligase. The ligation mixture is transfected into competent E. coli and selected for ampicillin resistance. Plasmid DNA is isolated from the colonies and analyzed by restriction analysis for proper orientation of the HBcAg/CETP/HBcAg sequences in the pMON3360B vector. A plasmid with the correct orientation is designated pMON3360B-HBcAg-CETP. Plasmid pMON3360B-HBcAg-CETP is purified using Promega Maxiprep™ protocols from 400 ml cultures.

BHK/VP16 hamster kidney cells are plated at about 3×10⁵ cells per 60 mm culture dish 24 hours before transfection in growth medium consisting of DMEM/5% fetal bovine sera (Life Technologies). Ten micrograms of plasmid pMON336OB-HBcAg-CETP and 1 μg of plasmid pMON1118 are transfected into the cells using LipofectAmine™ (Life Technologies) as recommended by the manufacturer. Two days after tranfection, the cells are treated with trypsin/EDTA (Life Technologies) and plated in ten 100 mm dishes in growth medium containing hygromycin (Sigma). In about two weeks, surviving colonies are isolated using filter paper and expanded and assayed for expression of the HBcAg/CETP/HBcAg fusion protein.

### Example 7

### Construction of DNA Vaccine Vectors Capable of In Vivo Expression of HBcAg/CETP/HBcAg Fusion Proteins

A. Construction of pcDNAI/Amp-HBcAg/CETP/HBcAg-1 and pcDNAI/Amp-HBcAg/CETP/HBcAg-2

Vector pcDNAI/Amp (Invitrogen Corp., San Diego, Calif.) is a derivative of vector pcDNAI and its parent vector pCDM8. All three vectors have the following features: Enhancer-promoter sequences from the immediate early gene of human cytomegalovirus (CMV) for high-level constitutive expression; SV40 poly(A) transcription termination and RNA processing signals to enhance mRNA stability, a versatile multiple cloning site to permit unidirectional or bidirectional cloning of inserts; and a ColE1 origin of replication for growth in E. coli. Vector pcDNAI/Amp also contains bacteriophage T7 and SP6 promoters flanking an expanded multiple cloning site, and an ampicillin resistance gene to facilitate growth and selection in most E. coli strains. These vectors can be used for high-level constitutive expression of recombinant proteins, including cytoplasmic proteins, transcription factors, viral proteins, cell surface receptors, and secreted proteins, in a variety of mammalian cells.

Vector pcDNAI/Amp can be linearized at any of a variety of positions in the multiple cloning site downstream from the pCMV promoter for insertion of heterologous genes. A blunt-ended DNA segment encoding HBcAg/CETP/HBcAg is prepared as described in Example 5 by digesting pProEx1-ABC-XYZ with NcoI and HindIII, and treating with Klenow enzyme in the presence of all four nucleotide triphosphates to make the 5′ overhanging ends blunt. This fragment is purified from an agarose gel. Vector pcDNAI/Amp is digested with EcoRV to leave blunt ends, treated with shrimp alkaline phosphatase to remove 5′ terminal phosphates, and ligated to the purified fragment encoding HBcAg/CETP/HBcAg. The resulting DNA is transformed into E. coli DH10B, and the resulting colonies are screened for plasmids containing the HBcAg/CETP/HBcAg inserted in the proper orientation downstream from the CMV promoter. The desired plasmid is designated pcDNAI/Amp-HBcAg/CETP/HBcAg-1.

A similar plasmid is also prepared by inserting the blunt-ended HBcAg/CETP/HBcAg into pcDNAI/Amp that is treated with BamHI, XbaI, and Klenow enzyme, to fill in the ends, to generate pcDNAI/Amp-HBcAg/CETP/HBcAg-2. This plasmid differs from pcDNAI/Amp-HBcAg/CETP/HBcAg-1 by removal of the large central portion of the multiple cloning site flanked by the CMV promoter and the SV40 poly(A) regions.

B. Construction of pCMV-SPORT-HBcAg/CETP/HBcAg

Plasmid pCMV-SPORT-β-gal (Life Technologies, Rockville, Md.) contains the E. coli β-galactosidase gene cloned as a NotI fragment into pCMV-SPORT1. These vectors contain a CMV promoter, an SV40 poly(A) site, an ampicillin resistance gene, and an E. coli plasmid origin of replication. Plasmid pCMV-SPORT-β-gal is commonly used as a reporter vector to monitor transfection efficiency. The plasmid PCMV-SPORT HBcAg/CETP/HBcAg is prepared using similar procedures describe above in subsection A. Plasmid pCMV-SPORT-β-gal is linearized with NotI, treated with Klenow to fill in the ends, and with shrimp alkaline phosphatase to remove 5′ terminal phosphates. The linearized, phosphatase-treated vector is then ligated to the blunt end linear fragment from above encoding HBcAg/CETP/HBcAg. The ligation mixtures are transformed into E. coli DH10B, and the resulting plasmids screened for the proper orientation of the insert into the vector.

Plasmids pCMV-SPORT-β-gal and pGreen Lantern-1 (Life Technologies, Rockville, Md.) both contain reporter genes under the control of the CMV promoter and are suitable as control vectors for use in mammalian transfection studies. Expression of β-galactosidase activity is easily monitored by in situ staining of prokaryotic or eukaryotic cells with the chromogenic substrate X-gal. Plasmid pGreen Lantern-1 contains a mutated form of the gene for Green Fluorescent Protein (GFP) from Aequorea victoria jellyfish. GFP requires no substrates for visualization, and can be monitored in living as well as fixed cells, and in whole animals, by fluorescence microscopy using FITC filters.

### Example 8

### Preparation of Purified DNA Template

Briefly, cultures of E. coli harboring plasmids are grown up in TB or LB media. [See, J. Sambrook et al., Molecular Cloning, 2nd ed., Cold Spring Harbor Press, Cold Spring Harbor N.Y., 8-23 (1989).] Plasmid DNA is released from pelleted cells by an alkaline lysis method using the reagents supplied in a commercial anion exchange purification kit (Qiagen GmbH, Hilden, Germany). The recovered lysate is purified on P-2500 columns (Qiagen), precipitated with isopropanol, solubilized in Tris-EDTA, reprecipitated in NaCl ethanol, and finally resuspended in sterile endotoxin-free phosphate buffered saline (PBS; Sigma Chemical, St. Louis, Mo.). Plasmid DNA is stored at −20 degrees C. The bulk product is tested for the presence of sterility and the presence of contaminating DNA, RNA, protein, and endotoxin according to established protocols [reviewed in M. Marquet et al., BioPharm, 10(5): 42-50 (1997)].

### Example 9

### In Vivo Expression of DNA Encoding HBcAg/CETP/HBcAg Fusion Proteins Injected Directly into the Muscles of Mice

Endotoxin-free samples of plasmids pcDNAI/Amp-HBcAg/CETP/HBcAg-1 and -2, pCMV-SPORT-HBcAg/CETP/HBcAg, pCMV-SPORT-β-gal, and pGreen Lantern-1 are prepared as described in the preceding examples. The quadriceps muscles of mice are injected with 100 μg of one of the plasmids listed above, and the muscle tissue at the site of injection is assayed for activity of the β-gal or GFP reporter gene or expression of the HBcAg/CETP/HBcAg fusion protein after a suitable maintenance time.

A. Light microscopy

The quadriceps muscles of mice immunized by to the plasmid DNA injections, are removed in their entirety, cross-sectioned, and histochemically-stained with X-gal for β-gal activity [Wolff et al., Science, 247:1465-1468, 1990]. Only those tissues exposed to pCMV-SPORT-β-gal produce the insoluble precipitate that is the indigo blue product resulting from cleavage of X-gal by the expressed β-galactosidase protein. Similarly, samples expressing GFP when viewed by light microscopy using FITC filters, show a dramatic fluorescent signal with an excitation peak of 490 nm. [R. Heim, et al., Nature, 373: 663 (1995).] Samples expressing the fusion protein do not fluoresce under these conditions nor react with X-gal.

B. Immunofluorescence

Muscle samples expressing the HBcAg/CETP/HBcAg fusion protein are detected by immunofluorescence with primary antibodies directed against HBcAg or CETP, and any of a number of well-known secondary antibodies (e.g. FITC- or Rhodamine-conjugated secondary antibodies). Established immunofluorescence protocols are widely known in the art, and many of the reagents and methods are available from commercial sources.

C. Immunoblotting

Muscle extracts are prepared by mincing quadriceps tissue in a microcentrifuge tube containing lysis buffer (20 mM Tris, pH 7.4, 2 MM MgCl₂, and 0.1% Triton X-100) and grinding with a plastic pestle until homogenized. Protein samples are analyzed on SDS-PAGE gels and electrophoretically-transferred to Immobilon-P or nitrocellulose membranes, using standard protocols. [See, J. Sambrook et al., Molecular Cloning, 2nd ed., Cold Spring Harbor Press, Cold Spring Harbor N.Y., 8-23 (1989).]. Primary antibodies directed against β-galactosidase, GFP, HBcAg, and CETP obtained from commercial sources are used to detect the expression products resulting from injection of plasmid DNAs into the muscle tissue. Many secondary antibodies are available from a wide variety of commercial sources that are chemically conjugated to a reporter enzymes. Commercially available examples include alkaline phosphatase-conjugated anti-rabbit, anti-mouse, or anti-human IgG. Secondary antibodies chemically conjugated to horse radish peroxidase or to β-galactosidase are also widely available. Muscle extracts expressing GFP are detected with anti-GFP antibodies, those expressing β-gal detected with anti-β-gal antibodies, and those expressing the HBcAg/CETP/HBcAg fusion protein are detected with antibodies directed against HBcAg or CETP.

D. PCR analysis

Muscle extracts are also analyzed to detect presence of the injected plasmid DNAs by polymerase chain reaction techniques. Briefly, muscle tissues taken from the site of injection are homogenized in lysis buffer, template DNA is prepared, and used in reactions containing appropriate mixes of primers and a thermostable polymerase. Primers lying within the ampicillin resistance gene, common to all vectors, amplify a DNA fragment in all tissues that take up and maintain the injected plasmid DNA. Primer sets specific for each gene, GFP, β-gal, HBcAg, and CETP, amplify uniquely-sized fragments, if the plasmid insert is intact within plasmids maintained within the cells. PCR can be used to monitor the long term stability of the plasmid within muscle tissues and aid in transcription and expression studies, if expression levels monitored by immunoblotting or microscopic techniques are low or nonexistent. PCR can also be used to determine whether the plasmids integrate into the host chromosome or are lost by passive diffusion.

E. Antibody Production

Mice immunized as discussed above produce antibodies that immunoreact with CETP in ELISA assays as discussed before. Similarly immunized rabbits also produce anti-CETP antibodies, which antibodies cause a lessening in the transfer of cholesteryl esters from HDL, and also an increase in the HDL particle concentration in blood plasma.

### Example 10

### In Vivo Expression of DNA Encoding Rabbit CETP Injected Directly into the Muscles of Mice

A. PCR Amplification of Rabbit CETP

Rabbit CETP cDNA (SEQ ID NO: 27) [Nagashima et al., J. Lipid Res., 29:1643-1649 (1988) or Kotake et al., Ibid, 37:599-605 (1996)] is obtained as described. PCR Primer N, below is designed to amplify the 5′ end of rabbit CETP and place an NotI (GC′GGCC,GC) and an NcoI (C′CATG,G) site in the correct reading frame at the natural ATG start condon immediately preceding the GCC condon at position +1 in SEQ ID NO:27. In each of the sequences shown below, only the coding strand is shown, with the bases removed after cleavage by restriction enzymes not being shown, and synthetic sequences being shown in lower case.

| Primer N: | |
|---|---|
| 5′ ggccgcccatgGCCTGTCCCAAAGGCGCCTCCTA | (SEQ ID |
| CGAGGCT 3′ | NO:51) |

Primer M, below, is designed to amplify the 3′ end of the rabbit CETP and place a TAA stop condon, a HindIII (A′ACGT,T) and NotI site (GC′GGCC,GC) following the amino acid residue 497 (Ser) immediately preceding the TAG stop codon ending at position +1494 of SEQ ID NO:27

| Primer M | |
|---|---|
| 5′ ggccgcacgttttaCTAGCTCAGGCTCTGCAGGA | (SEQ ID |
| AATCCACCAGCAGGTG | NO:52) |

A DNA fragment encoding the full-length rabbit CETP with modified 5′ and 3′ ends is amplified using the above cDNA as a template in the presence of primer M and primer N under the standard polymerase chain reaction conditions recommended by the manufacturer of the GeneAmp PCR reagent kit (Perkin Elmer Cetus, Norwalk, Conn.). The amplified DNA is then cleaved with NotI and fractionated by size on an agarose gel. Full-length rabbit CETP DNA is purified as described above.

B. Insertion of Rabbit CETP DNA into pGEM-5Zf(+), pCR II, and pProEx1

The full-length rabbit CETP with NotI-compatible ends is inserted into a plasmid cloning vector, such as pGEM™-5Zf(+) (Promega Corporation, Madison, Wis.), which has a unique NotI site in the polylinker region within the lacZalpha peptide region. Recombinants are selected by blue/white color screening as recommended by the vendor. An alternate procedure is to clone the PCR product containing the full-length rabbit CETP directly into a TA cloning vector, such as pCR{fourth root} II (Invitrogen Corporation, San Diego, Calif.). DNA sequencing is carried out to confirm the identity of the nucleotide sequence encoding the rabbit CETP. In either case, the full-length rabbit CETP DNA can be released from the vector as a fragment with NotI-compatible ends, or as an NcoI-HindIII fragment.

C. Expression of Rabbit CETP in E. coli, Insect Cells, and Mammalian Cells

The rabbit CETP is inserted into pProEx1 as an NcoI-HindIII fragment as described above in Examples 3B and 3C for the DNA encoding the HbcAg/CETP/HbcAg chimeric fusion protein. Expression of the rabbit CETP protein in E. coli is also carried out as described above in Example 4. The rabbit CETP protein is then purified from E. coli, to confirm the biological activity of the isolated protein.

The DNA encoding rabbit CETP is purified and inserted into plasmid pFastBac1 as described in Example 5. Recombinant baculoviruses are generated and used to express rabbit CETP in infected insect cells.

The DNA encoding rabbit CETP is purified and inserted into pMON3327 as described in Example 6. BHK/VP16 cells are transfected with the resulting plasmid and surviving colonies are expanded and assayed for expression of the rabbit CETP.

D. Insertion of the Rabbit CETP gene into DNA Vaccine Vectors

The rabbit CETP gene (rCETP) is inserted as a blunt-ended fragment into vector pcDNAI/Amp as described in Example 7A. The rabbit CETP gene is also inserted into pCMV-SPORT as an NotI fragment as described in Example 7B. Both plasmids direct expression of rabbit CETP in mammalian cells under the control of the CMV promoter.

Mammalian cells are transfected with pcDNAI/Amp-rCETP or pCMV-SPORT-rCETP. Expression of rabbit CETP is assayed by immunofluorescence techniques as described in Example 9B and by immunoblotting techniques, as described in Example 9C.

Once rabbit CETP is detectable and expression levels are suitably high in transfected mammalian cell lines to provide assurance of expression, injection of pcDNAI/Amp-rCETP or pCMV-SPORT-rCETP into muscle tissues of animals containing CETP in their blood, such as Guinea pigs for example, is carried out and monitored as described in Examples 9A-E.

The foregoing description and the examples are intended as illustrative and are not to be taken as limiting. Still other variations within the spirit and scope of this invention are possible and will readily present themselves to those skilled in the art.

1. A process for producing antibodies to cholesteryl ester transfer protein (CETP) in a mammal that comprises the steps of:
(a) immunizing said mammal with an inoculum containing a vehicle in which is dissolved or dispersed a recombinant DNA molecule comprising a DNA sequence that contains (i) a sequence encoding a CETP immunogen linked to (ii) a promoter sequence that controls the expression of said CETP immunogen DNA sequence in said mammal, said CETP immunogen being an immunogenic polypeptide having a CETP amino acid residue sequence, said immunization providing an amount of said recombinant DNA molecule sufficient to induce antibodies to CETP; and
(b) maintaining said immunized mammal for a time period sufficient for the production of antibodies that bind to CETP.

2. The process of claim 1 wherein the blood of said mammal contains CETP.

3. A process for increasing the concentration of HDL cholesterol in the blood of a mammal whose blood contains cholesterol ester transfer protein (CETP) that comprises the steps of:
(a) immunizing said mammal with an inoculum containing a vehicle in which is dissolved or dispersed a recombinant DNA molecule comprising a DNA sequence that contains (i) a sequence encoding a CETP immunogen linked to (ii) a promoter sequence that controls the expression of said CETP immunogen DNA sequence in said mammal, said CETP immunogen being an immunogenic polypeptide having a CETP amino acid residue sequence, said immunization providing an amount of said recombinant DNA molecule sufficient to induce antibodies to CETP; and
(b) maintaining said immunized mammal for a time period sufficient for said CETP immunogen to be expressed and for the production of antibodies that bind to CETP and lessen the transfer of cholesteryl esters from HDL.

4. The process according to claim 3 wherein said immunizing step is repeated.

5. The process according to claim 3 wherein said immunizing step is repeated at intervals of about 3 to about 6 months until the HDL cholesterol value in the blood of said mammal is increased by about 10 percent or more relative to the HDL cholesterol value prior to said first immunization step.

6. The process according to claim 3 wherein said recombinant DNA molecule encodes human CETP as said immunogenic polypeptide.

7. The process according to claim 3 wherein said recombinant DNA molecule encodes rabbit CETP as said immunogenic polypeptide.

8. The process according to claim 3 wherein said encoded CETP immunogen comprises an immunogenic polypeptide fused to an exogenous antigenic carrier polypeptide.

9. The process according to claim 8 wherein said exogenous antigenic carrier polypeptide is selected from the group consisting of hepatitis B core protein, tetanus toxoid, and diphtheria toxoid.

10. The process according to claim 9 wherein said recombinant DNA molecule encodes a fusion protein in which said exogenous antigenic carrier is fused to the carboxy-terminus of said immunogenic polypeptide.

11. The process according to claim 8 wherein the carboxy-terminus of said encoded exogenous antigenic carrier is fused to the amino-terminus of said encoded immunogenic polypeptide.

12. The process according to claim 8 wherein said encoded exogenous antigenic carrier is fused to both the amino-terminus and carboxy-terminus of said encoded immunogenic polypeptide.

13. The process according to claim 12 wherein said encoded fusion protein is comprised of an immunogenic polypeptide having a length of about 10 to about 30 amino acid residues that are fused to an amino-terminal flanking sequence and a carboxy-terminal flanking sequence, wherein
(a) said amino-terminal flanking sequence consists essentially of about 10 to about 20 amino acid residues having an amino acid residue sequence of the hepatitis B core protein (HBcAg) from about position 1 to about position 35, and said carboxy-terminal sequence consists essentially of about 120 to about 160 amino acid residues having an amino acid residue sequence of HBcAg from about position 10 about position 183, or
(b) said amino-terminal flanking sequence consists essentially of about 70 to about 90 residues having the amino acid residue sequence of HBcAg from about position 1 to about position 90, and said carboxy-terminal flanking sequence consists essentially of about 65 to about 85 amino acid residues having the amino acid residue sequence of HBcAg from about position 80 to about position 183.

14. The process according to claim 13 wherein the number of amino acid residues present in said encoded immunogenic polypeptide is about equal in number to the number of amino acid residues absent from said HBcAg amino acid residue sequence between the carboxy-terminal residue position of said amino-terminal flanking sequence and the amino-terminal residue of said carboxy-terminal flanking sequence.

15. The process according to claim 3 wherein said encoded immunogenic polypeptide has the amino acid residue sequence of SEQ ID NOs:29 or 50.

16. The process according to claim 3 wherein said immunization is carried out by injecting said inoculum into muscle or skin of said mammal.

17. An inoculum that comprises a recombinant DNA molecule comprising a DNA sequence that contains (i) a sequence encoding a CETP immunogen linked to (ii) a promoter sequence that controls the expression of said CETP immunogen DNA sequence in a mammal, said recombinant DNA molecule being dissolved or dispersed in an effective amount in a vehicle.

18. The inoculum of claim 17 wherein the concentration of said DNA encoding said CETP immunogen is about 0.05 μg/ml to about 20 mg/ml.

19. The inoculum of claim 17 wherein said vehicle is phosphate-buffered saline.

20. The inoculum of claim 17 wherein said vehicle is isotonic sucrose.

21. The inoculum of claim 17 wherein said DNA is complexed with liposomes.
